(19) United States
(12) Patent Application Publication
(10) Pub. No.: US 2009/0124600 A1
(43) Pub. Date: May 14, 2009

(54) N-Alkyl-Azacycloalkyl NMDA/NR2B Antagonists

(76) Inventor: Mark E. Layton, Harleysville, PA (US)

Correspondence Address: MERCK AND CO., INC, P O BOX 2000, RAHWAY, NJ, 07065-0907, US

(21) Appl. No.: 11/918,336

(22) PCT Filed: Apr. 14, 2006
(86) PCT No.: PCT/US06/14139

§ 371 Date: Oct. 10, 2007

Related U.S. Application Data
(60) Provisional application No. 60/672,693, filed on Apr. 19, 2005.

(51) Int. Cl. A61K 31/55; C07D 487/04; A61K 31/519

(52) U.S. Cl. 514/217.06; 544/262; 540/600; 514/262.1

(57) ABSTRACT

Compounds represented by Formula (I): and/or pharmaceutically acceptable salts, individual enantiomers and stereoisomers thereof, are effective as NMDA/NR2B antagonists useful for treating conditions such as pain, Parkinson's disease, Alzheimer's disease, epilepsy, depression, anxiety, ischemic brain injury including stroke.

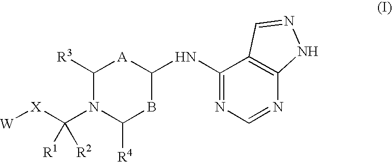

### FIELD OF THE INVENTION

This invention relates to N-alkyl-azacycloalkyl compounds. In particular, this invention relates to N-alkyl-azacycloalkyl compounds that are NMDA/NR2B antagonists useful for the treatment of neurological conditions such as pain, Parkinson's disease, Alzheimer's disease, epilepsy, depression, anxiety, ischemic brain injury including stroke, and other conditions.

### BACKGROUND OF THE INVENTION

Ions such as glutamate play a key role in processes related to chronic pain and pain-associated neurotoxicity—primarily by acting through N-methyl-D-aspartate (“NMDA”) receptors. Thus, inhibition of such action—by employing ion channel antagonists, particularly NMDA antagonists—can be beneficial in the treatment and control of Parkinson's disease and pain.

NMDA receptors are heteromeric assemblies of subunits, of which two major subunit families designated NR1 and NR2 have been cloned. Without being bound by theory, it is generally believed that the various functional NMDA receptors in the mammalian central nervous system (“CNS”) are only formed by combinations of NR1 and NR2 subunits, which respectively express glycine and glutamate recognition sites. The NR2 subunit family is in turn divided into four individual subunit types: NR2A, NR2B, NR2C, and NR2D. T. Ishii, et al., J. Biol. Chem., 268:2836-2843 (1993), and D. J. Laurie et al., Mol. Brain Res., 51:23-32 (1997) describe how the various resulting combinations produce a variety of NMDA receptors differing in physiological and pharmacological properties such as ion gating properties, magnesium sensitivity, pharmacological profile, as well as in anatomical distribution.

For example, while NR1 is found throughout the brain, NR2 subunits are differentially distributed. In particular, it is believed that the distribution map for NR2B lowers the probability of side effects while treating Parkinson's disease or pain. Thus, it would be desirable to provide novel NMDA antagonists that target the NR2B receptor.

### SUMMARY OF THE INVENTION

The present invention relates to compounds represented by Formula I:

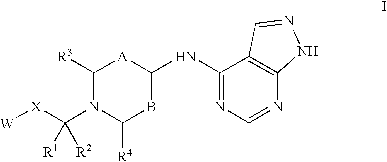

(wherein W, X, A, B, R¹, R², R³ and R⁴ are described herein) or pharmaceutically acceptable salts thereof. This invention further provides methods to treat and prevent neurological conditions, including pain, Parkinson's disease, Alzheimer's disease, epilepsy, depression, anxiety, ischemic brain injury including stroke, and other conditions, utilizing the present compounds and compositions.

### DETAILED DESCRIPTION OF THE INVENTION

The compounds of this invention are represented by Formula I:

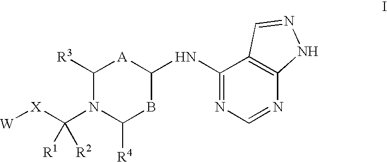

and pharmaceutically acceptable salts and individual enantiomers and stereoisomers thereof, wherein:
W is aryl or heteroaryl, wherein said aryl or heteroaryl is optionally substituted with 1-5 substituents independently selected from the group consisting of halogen, C₃₋₆ cycloalkyl, cyano, C₁₋₄ alkoxy, and C₁₋₆ alkyl, wherein said alkoxy is optionally substituted with one or more halogen, and said alkyl is optionally substituted with one or more substituents selected from the group consisting of hydrogen, halogen and hydroxyl;
X is absent or is selected from the group consisting of C₁₋₄alkoxy and C₁₋₃alkyl optionally substituted with one or more substituents selected from the group consisting of hydrogen, halogen, hydroxyl, (═O), and cyano;
A is a bond or C₁₋₃ alkyl, optionally substituted with one or more substituents selected from the group consisting of hydrogen, halogen, hydroxyl, and C₁₋₃ alkyl, wherein said alkyl is optionally substituted with one or more substituents selected from the group consisting of halogen, hydroxyl, C₁₋₄ alkoxy, and cyano;
B is C₁ alkyl, optionally substituted with one or more substituents selected from the group consisting of hydrogen, halogen, hydroxyl, and C₁₋₃ alkyl, wherein said alkyl is optionally substituted with one or more substituents selected from the group consisting of halogen, hydroxyl, C₁₋₄ alkoxy, and cyano, where a ring is formed comprising A and B, where an individual carbon atom in A and an individual carbon atom in B optionally can join to bridge said ring;
R¹ and R² each is independently selected from the group consisting of hydrogen and C₁₋₃ alkyl; and
R³ and R⁴ each is independently selected from the group consisting of hydrogen, hydroxyl, cyano, and C₁₋₃ alkyl, wherein said alkyl is optionally substituted with one or more substituents selected from the group consisting of halogen, hydroxyl, C₁₋₄ alkoxy, and cyano,
where R³ and R⁴, along with the ring to which they are attached, optionally can join to form a bridged cycloalkyl.

One embodiment of the present invention provides a compound described by the chemical Formula (I), and/or a pharmaceutically acceptable salt, individual enantiomer and stereoisomer thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;

X is selected from the group consisting of C₁₋₄alkoxy and C₁₋₃ alkyl optionally substituted with one or more substituents selected from the group consisting of hydrogen, halogen, hydroxyl and (═O);

R¹ and R² each is independently selected from the group consisting of hydrogen and C₁₋₃ alkyl;

R³ and R⁴ each is independently hydrogen;

A is a bond or C₁₋₃ alkyl, optionally substituted with one or more substituents selected from the group consisting of hydrogen and halogen; and

B is C₁alkyl, optionally substituted with one or more substituents selected from the group consisting of hydrogen and halogen, where a ring is formed comprising A and B, where an individual carbon atom in A and an individual carbon atom in B optionally can join to bridge said ring.

A further embodiment of the present invention includes compounds represented by Formula Ia:

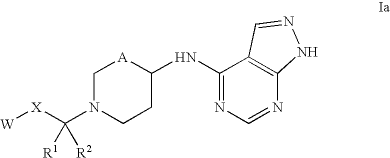

and/or pharmaceutically acceptable salts, individual enantiomers and stereoisomers thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;

X is C₁₋₃ alkyl optionally substituted with one or more substituents selected from the group consisting of hydrogen, halogen, hydroxyl and (═O);

R¹ and R² each is independently selected from the group consisting of hydrogen and C₁₋₃ alkyl; and

A is a bond or C₁₋₃ alkyl, optionally substituted with one or more substituents selected from the group consisting of hydrogen and halogen.

Another embodiment of this invention includes compounds represented by Formula Ib:

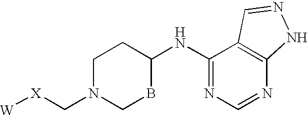

and/or pharmaceutically acceptable salts, individual enantiomers and stereoisomers thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;

X is C₁₋₃ alkyl optionally substituted with one or more substituents selected from the group consisting of hydrogen, halogen, hydroxyl and (═O); and

B is C₁alkyl, optionally substituted with one or more substituents selected from the group consisting of hydrogen and halogen.

Another embodiment of this invention includes compounds represented by Formula Ic:

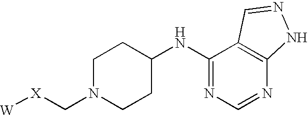

and/or pharmaceutically acceptable salts, individual enantiomers and stereoisomers thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl; and

X is C₁₋₃ alkyl optionally substituted with one or more substituents selected from the group consisting of hydrogen, halogen, hydroxyl and (═O).

Another embodiment of this invention includes compounds represented by Formula Id:

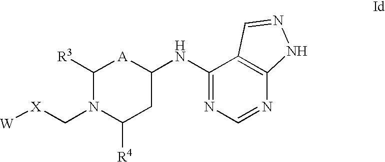

and/or pharmaceutically acceptable salts, individual enantiomers and stereoisomers thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;

X is C₁₋₃ alkyl optionally substituted with one or more substituents selected from the group consisting of hydrogen and halogen;

A is a bond or C₁₋₃ alkyl, optionally substituted with one or more substituents selected from the group consisting of hydrogen and halogen; and

R³ and R⁴ each is independently selected from the group consisting of hydrogen, hydroxyl, cyano, and C₁₋₃ alkyl, wherein said alkyl is optionally substituted with one or more substituents selected from the group consisting of halogen, hydroxyl, C₁₋₄ alkoxy, and cyano,

where R³ and R⁴, along with the ring to which they are attached, can join to form a bridged cycloalkyl.

Another embodiment of this invention includes compounds represented by Formula Ie:

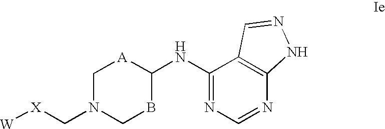

and/or pharmaceutically acceptable salts, individual enantiomers and stereoisomers thereof, wherein:

W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;

X is C₁₋₃ alkyl optionally substituted with one or more substituents selected from the group consisting of hydrogen, halogen, hydroxyl and (═O);

A is a bond or C₁₋₃ alkyl, optionally substituted with one or more substituents selected from the group consisting of hydrogen and halogen; and

B is C₁alkyl, optionally substituted with one or more substituents selected from the group consisting of hydrogen and halogen, where a ring is formed comprising A and B, where an individual carbon atom in A and an individual carbon atom in B optionally can join to bridge said ring.

As used herein, “alkyl” as well as other groups having the prefix “alk” such as, for example, alkane, alkanoyl, alkenyl, and alkynyl means carbon chains that may be linear or branched or combinations thereof. Examples of suitable alkyl groups include methyl, ethyl, propyl, isopropyl, butyl, sec- and tert-butyl, pentyl, hexyl, and heptyl. Thus, C₁₋₆alkyl is defined to identify the group as having 1, 2, 3, 4, 5 or 6 carbons in a linear or branched arrangement, such that C₁₋₆alkyl includes methyl, ethyl, n-propyl, iso-propyl, n-butyl, iso-butyl, tert-butyl, pentyl and hexyl. Similarly, C₁₋₄alkyl is defined to identify the group as having 1, 2, 3 or 4 carbons in a linear or branched arrangement, such that C₁₋₄alkyl specifically includes methyl, ethyl, n-propyl, iso-propyl, n-butyl, iso-butyl and tert-butyl. Likewise, C₀, as in C₀alkyl, is a hydrogen atom substituent when the alkyl is a terminal group and is a direct bond when the alkyl is a bridging group. “Alkenyl”, “alkynyl” and other like terms include carbon chains containing at least one unsaturated C—C bond.

The term “alkoxy” as used herein, alone or in combination, includes alkyl ether groups, wherein the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxyethane (also referred to as ‘ethyl methyl ether’).

As used herein, the term “cycloalkyl” is intended to mean carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, and 1,2,3,4-tetrahydronaphalene.

As used herein, “aryl” is intended to mean any stable monocyclic or bicyclic carbon ring of up to 7 members in each ring, wherein at least one ring is aromatic. Examples of such aryl elements include phenyl, napthyl and tolyl.

As used herein, “bridged cycloalkyl” is intended to mean two or more cycloalkyl groups, heterocycloalkyl groups, or a combination thereof joined via adjacent or non-adjacent atoms. Examples of such bridged cycloalkyl groups include 8-azabicyclo[3.2.1]oct-3-yl and 3-azabicyclo[3.1.0]hex-6-yl.

The term “heteroaryl”, as used herein except where noted, is intended to mean a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, pyridine, pyrimidine, pyrazine, thiophene, oxazole, thiazole, triazole, thiadiazole, oxadiazole, pyrrole, 1,2,4-oxadiazole, 1,3,4-oxadiazole, 1,2,4-thiadiazole, 1,3,4-thiadiazole, and 1,2,4-triazole.

The term “hetero,” unless specifically stated otherwise, includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heterocycloalkyls include azetidinyl, pyrrolidinyl, piperidinyl, piperazinyl, morpholinyl, tetrahydrofuranyl, imidazolinyl, pyrrolidin-2-one, piperidin-2-one, and thiomorpholinyl.

In the structures depicted throughout this application a hydrogen atom on an unsubstituted nitrogen atom may be either expressly shown or implicit.

The same convention also applies to all generic structures and structures depicting individual species. Of course, nitrogen atoms may also be substituted with atoms and/or moieties other than hydrogen, as set forth elsewhere in this application.

As appreciated by those of skill in the art, the terms halo or halogen as used herein is intended to include chloro, fluoro, bromo and iodo.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the alkyl and the aryl groups are optionally substituted. If only one of the multiple moieties is optionally substituted then it will be specifically recited such as “an alkylaryl, the aryl optionally substituted with halogen or hydroxyl.”

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. Formula I (including Formulas Ia, Ib, Ic, Id, and Ie) is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The subject or patient treated in the present methods is generally a mammal such as a human being, male or female, in whom antagonism of NMDA/NR2B receptor activity is desired. The subject or patient treated in the present methods can also be an animal such as a dog, cat, horse, pig, or cow. The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The pharmaceutical compositions of the present invention comprise a compound represented by Formula I (and/or pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present invention is further directed to a method for the manufacture of a medicament for the antagonism of NMDA/NR2B receptor activity in humans and animals comprising combining a compound of the present invention with a pharmaceutical carrier or diluent.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, and/or pharmaceutically acceptable salt(s) thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.5 mg to about 5 g of the active ingredient and each cachet or capsule preferably containing from about 0.5 mg to about 5 g of the active ingredient.

The pharmaceutical compositions of the present invention comprise a compound represented by Formula I (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the alt of pharmacy.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage: thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, and/or pharmaceutically acceptable salts thereof, may also be prepared in powder or liquid concentrate form.

The utility of the compounds in accordance with the present invention as antagonists of NMDA/NR2B receptor activity may be demonstrated by methodology known in the art. Inhibition of the binding to NMDA receptors and functional antagonism of calcium efflux through NMDA channels were determined as follows:

### Cell-Based Functional Assay to Determine IC₅₀ of NR2B Antagonists

The ability of selected compounds to inhibit NR1a/NR2B NMDA receptor, as measured by NR1a/NR2B receptor-mediated Ca²⁺ influx, was assessed by the following calcium flux assay procedure:

NR1a/NR2B receptor transfected L(tk-) cells were plated in 96-well format at 3×10⁴ cells per well and grown for one to two days in normal growth medium (Dulbeccos MEM with Na pyruvate, 4500 mg glucose, pen/strep, glutamine, 10% FCS and 0.5 mg/mL geneticin). NR1a/NR2B-expression in these cells was induced by the addition of 4-20 nM dexamethasone in the presence of 500 μM ketamine for 16-24 hours. Solutions of NR2B antagonists were prepared in DMSO and serially diluted with DMSO to yield 10 solutions differing by 3-fold in concentration. A 96-well drug plate was prepared by diluting the DMSO solution 250-fold into assay buffer (Hanks Balanced Salt Solution (HBSS) Mg²⁺ free (Gibco #14175-079) containing 20 mM HEPES, 2 mM CaCl₂, 0.1% BSA and 250 μM Probenecid (Sigma # P-8761)). After induction, the cells were washed twice (Labsystem cell washer, 3 fold dilutions leaving 100 μL) with assay buffer and loaded with 4 μM of the calcium fluorescence indicator fluo-3 AM (Molecular Probes # P-1241) in assay buffer containing Pluronic F-127 (Molecular Probes # P-3000) and 10 μM ketamine at 37° C. for one hour. The cells were then washed eight times with assay buffer leaving 100 μL of buffer in each well. Fluorescence intensity was immediately measured in a FLIPR (Fluorometric Imaging Plate Reader, Molecular Devices) using an excitation of 488 nm and emission at 530 nm. Five seconds after starting the recording of fluorescence intensity, 50 μL of agonist solution (40 μM glutamate/glycine, the final concentration 10 μM) was added and after one minute, when fluorescence signal was stable, 50 μL of NR2B antagonists and control solutions from the drug plate were added and the fluorescence intensity recorded for another 30 minutes. The IC₅₀ values were determined by a non-linear least squares fitting of the endpoint fluorescence values to Equation #1 below.

$$Endpoint Florescence = \frac{( Y max - Y min )}{1 + 9 {( [ Drug ] / {IC}_{50} )}^{nH}} + Y min \qquad Equation \#1$$

where, Ymin is average endpoint fluorescence of the control wells containing 1 μM of AMD-2 and Ymax is the average endpoint fluorescence of wells containing 0.1% DMSO in assay buffer.

### Binding Assay to Determine K_{I} NR2B Antagonists

The radioligand binding assay was performed at room temperature in 96-well microtiter plates with a final assay volume of 1.0 mL in 20 mM Hepes buffer (pH 7.4) containing 150 mM NaCl. Solutions of NR2B antagonists were prepared in DMSO and serially diluted with DMSO to yield 20 μL of each of 10 solutions differing by 3-fold in concentration. Non-specific binding (NSB) was assessed using AMD-1 (10 μM final concentration), and total binding (TB) was measured by addition of DMSO (2% final concentration). Membranes expressing NR1a/NR2B receptors (40 μM final concentration) and tritiated AMD-2 (1 nM final concentration) were added to all wells of the microtiter plate. After 3 hours of incubation at room temperature, samples are filtered through Packard GF/B filters (presoaked in 0.05% PEI, polyethyleninine Sigma P-3143) and washed 10 times with 1 mL of cold 20 mM Hepes buffer per wash. After vacuum drying of the filter plates, 40 μL of Packard Microscint-20 was added and bound radioactivity determined in a Packard TopCount. The apparent dissociation constant (K_{I}), the maximum percentage inhibition (% Iₘₐₓ), the minimum percentage inhibition (% Iₘᵢₙ) and the hill slope (nH) were determined by a non-linear least squares fitting the bound radioactivity (CPM bound) to Equation #2 below.

$$CPM Bound = \frac{( SB ) ( \% {I}_{m ax} - \% {I}_{m i n} ) / 100}{( 1 + {( [ Drug ] / ( {K}_{1} ( 1 + [ AMD \text{-} 2 ] / {K}_{D} ) ) )}^{nH} )} + NSB + ( SB ) ( 100 \text{-} \% {I}_{m ax} ) \text{/} 100 \qquad Equation \#2$$

where, K_{D} is the apparent dissociation constant for the radioligand for the receptor as determined by a hot saturation experiment and SB is the specifically bound radioactivity determined from the difference of TB and NSB control wells.

### Synthesis of AMD-1 and AMD-2 May be Accomplished According to the Following Reaction Schemes:

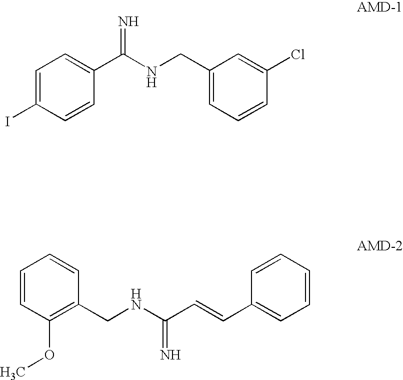

The precursor E for the synthesis of radiolabelled AMD-1 can be synthesized in accordance with the following procedure:

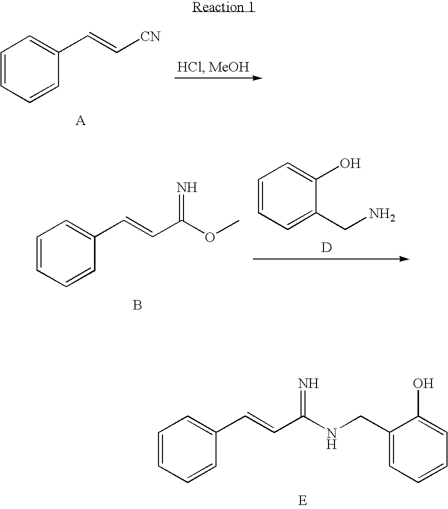

In accordance with the procedures of Reaction 1, hydrogen chloride is bubbled through a solution of cinnamonitrile A in methanol at room temperature. The volatiles are removed under reduced pressure and the resulting residue is triturated with ether and filtered to yield the intermediate imidate B. Imidate B is dissolved in methanol at ambient temperature, treated with amine D (commercially available from Acros Chemicals) at ambient temperature and stirred under argon. The volatiles are removed under reduced pressure and the residue purified by preparative HPLC or trituration with ether to afford amidine E.

Tritiated AMD-2 can be synthesized according to the following procedure:

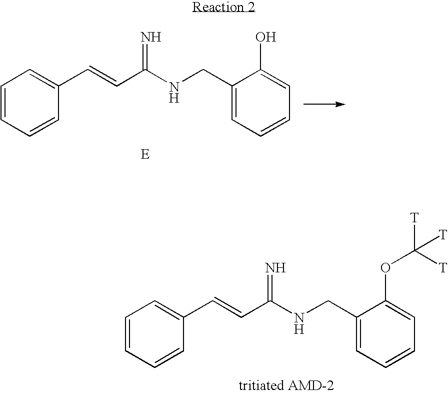

Tritiated AMD-2 was prepared by the following procedure, illustrated above in Reaction 2: The precursor E (2 mg, 0.008 mmol) dissolved in dimethylformamide (0.6 mL) and potassium carbonate (1.2 mg) for 1 h. High specific activity tritiated methyl iodide (50 mCi, 0.0006 mmol, in toluene 1 mL, commercially available from American Radiolabeled Chemicals, St. Louis, Mo.) was added at room temperature and stirred for 2 hours. The reaction mixture was filtered using a Whatman PTFE 0.45 μm syringeless filter device to remove any insoluble potassium carbonate, washed with Abs. ethanol (2 mL, commercially available from Pharmco), and the combined filtrates were concentrated to dryness at room temperature using a rotary evaporator, this also removed any unreacted tritiated methyl iodide. The residue was purified by HPLC chromatography on a Phenomenx Luna C8 semi-prep column (Luna 5 micro C8(2), 250×10.0 mm) using a gradient system of 20/80 acetonitrile/water with 0.1% trifluoroacetic acid to 100% acetonitrile with 0.1% trifluoroacetic acid in 20 min. Total activity of the product was 8 mCi. Further purification was effected by absorption onto a column (Waters Sep-Pak® PLUS C18 column, commercially available from Waters Corporation, Milford, Mass.) and elution with water followed by absolute ethanol. The product was diluted with absolute ethanol (10 mL) before submission for final analysis.

AMD-1 can be synthesized according to the general procedure described by C. F. Claiborne et al (Bioorganic & Med. Chem. Letters 13, 697-700 (2003).

Unlabelled AMD-2 is prepared as follows:

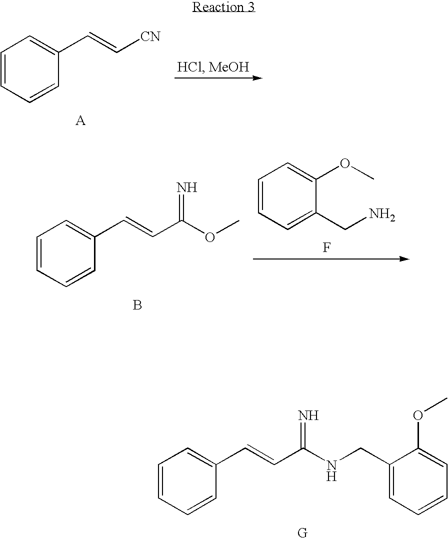

In accordance with Reaction 3, hydrogen chloride is bubbled through a solution of cinnamonitrile A in methanol at room temperature. The volatiles are removed under reduced pressure and the resulting residue is triturated with ether and filtered to yield the intermediate imidate B. Imidate B is dissolved in methanol at ambient temperature, treated with amine F at ambient temperature and stirred under argon. The volatiles are removed under reduced pressure and the residue purified by preparative HPLC or trituration with ether to afford amidine G.

The compounds of this invention exhibit IC₅₀ and K_{I} values of less than 50 μM in the functional and binding assays, respectively. It is advantageous that the IC₅₀ and K_{I} values be less than 5 μM in the functional and binding assays, respectively. It is more advantageous that the IC₅₀ and K, values be less than 1 μM in the functional and binding assays, respectively. It is still more advantageous that the IC₅₀ and K_{I} values be less than 0.1 μM in the functional and binding assays, respectively.

The present compounds are NMDA NR2B receptor antagonists, and as such are useful for treating, preventing, controlling, ameliorating or reducing the risk of diseases and disorders mediated through the NR2B receptor. Such diseases and disorders include, but are not limited to, neuropathic pain (such as postherpetic neuralgia, nerve injury, the “dynias”, e.g., vulvodynia, phantom limb pain, root avulsions, painful diabetic neuropathy, compressive mononeuropathy, ischemic neuropathy, painful traumatic mononeuropathy, or painful polyneuropathy), central pain syndromes (potentially caused by virtually any lesion at any level of the nervous system), and postsurgical pain syndromes (eg, postmastectomy syndrome, postthoracotomy syndrome, stump pain), bone and joint pain (osteoarthritis, rheumatoid arthritis, ankylosing spondylitis), repetitive motion pain, carpal tunnel syndrome, dental pain, cancer pain, myofascial pain (muscular injury, fibromyalgia), perioperative pain (general surgery, gynecological), chronic pain, dysmenorrhea, as well as pain associated with angina, and inflammatory pain of varied origins (e.g. osteoarthritis, rheumatoid arthritis, rheumatic disease, teno-synovitis and gout), headache, migraine and cluster headache, depression, anxiety, schizophrenia, stroke, traumatic brain injury, Alzheimer's disease, cerebral ischemia, spinal cord injury, cerebral vascular disease, Meniere's disease, vertigo, amyotrophic lateral sclerosis, Huntington's disease, sensorineural hearing loss, tinnitus, macular degeneration, glaucoma, neurological damage caused by epileptic seizures or by neurotoxin poisoning or by impairment of glucose and/or oxygen to the brain, vision loss caused by neurodegeneration of the visual pathway, Restless Leg Syndrome, multi-system atrophy, non-vascular headache, primary hyperalgesia, secondary hyperalgesia, primary allodynia, secondary allodynia, or other pain caused by central sensitization.

Compounds of Formula I are useful for treating or preventing movement disorders such as Parkinson's disease, dyskinesias (including the side effects accompanying normal doses of L-Dopa), tardive diskinesia, drug-induced parkinsonism, postencephalitic parkinsonism, progressive supranuclear palsy, multiple system atrophy, corticobasal degeneration, parkinsonian-ALS dementia complex, basal ganglia calcification, akinesia, akinetic-rigid syndrome, bradykinesia, dystonia, medication-induced parkinsonian, Gilles de la Tourette syndrome, Huntingon disease, tremor, chorea, myoclonus, tick disorder, and dystonia.

As used herein, the term “movement disorders” includes akinesias and akinetic-rigid syndromes, dyskinesias and medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor). Examples of “akinetic-rigid syndromes” include Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification. Examples of “dyskinesias” include tremor (including rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia).

Furthermore, compounds of Formula I may be used to treat substance abuse disorders, including by decreasing tolerance and/or dependence to opioid treatment of pain, and/or by treating withdrawal syndrome of e.g., alcohol, opioids, heroin, and cocaine. As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

In particular, compounds of structural formula I are useful for aiding in stopping consumption of tobacco and are useful in treating nicotine dependence and nicotine withdrawal. The compounds of formula I produce in consumers of nicotine, such as tobacco smokers, a total or partial abstinence from smoking. Further, withdrawal symptoms are lessened and the weight gain that generally accompanies quitting tobacco consumption is reduced or nonexistent. For smoking cessation, the compound of form I may be used in combination with a nicotine agonist or a partial nicotine agonist, or a monoamine oxidase inhibitor (MAOI), or another active ingredient demonstrating efficacy in aiding cessation of tobacco consumption; for example, an antidepressant such as bupropion, doxepine, ornortriptyline; or an anxiolytic such as buspirone or clonidine.

The compounds of this invention are also useful for treating or preventing HIV- and HIV treatment-induced neuropathy, chronic pelvic pain, neuroma pain, complex regional pain syndrome, chronic arthritic pain and related neuralgias, treating or preventing chronic lower back pain, and treating or preventing pain resulting from, or associated with, traumatic nerve injury, nerve compression or entrapment, postherpetic neuralgia, trigeminal neuralgia, diabetic neuropathy, cancer and chemotherapy.

It is understood that compounds of this invention can be administered at prophylactically effective dosage levels to prevent the above-recited conditions, as well as to prevent other conditions mediated through the NMDA NR2B receptor.

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other drugs or therapeutic agents, in addition to a compound of Formula I. Examples of other therapeutic agents or drugs that may be combined with a compound of Formula I, either administered separately or in the same pharmaceutical compositions, include, but are not limited to: (1) non-steroidal anti-inflammatory agents; (2) COX-2 inhibitors; (3) bradykinin B1 receptor antagonists; (4) sodium channel blockers and antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; (14) neutrophil inhibitory factor (NIF); (15) L-Dopa; (16) carbidopa; (17) levodopa/carbidopa; (18) dopamine agonists such as bromocriptine, pergolide, pramipexole, ropinirole; (19) anticholinergics; (20) amantadine; (21) carbidopa; (22) catechol O-methyltransferase (“COMT”) inhibitors such as entacapone and tolcapone; (23) Monoamine oxidase B (“MAO-B”) inhibitors; (24) opiate agonists or antagonists; (25) 5HT receptor agonists or antagonists; (26) NMDA receptor agonists or antagonists; (27) NK1 antagonists; (28) selective serotonin reuptake inhibitors (“SSRI”) and/or selective serotonin and norepinephrine reuptake inhibitors (“SSNRI”); (29) tricyclic antidepressant drugs, (30) norepinephrine modulators; (31) lithium; (32) valproate; (33) neurontin (gabapentin), (34) pregabalin, (35) antiplatelet agents, (36) VR1 antagonists, (37) COX-3 inhibitors, (38) antioxidants, (39) metal chelators, (40) CGRP antagonists, and (41) memantine.

Suitable antiparkinsonian agents of use in combination with deramcicline include levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, and dopamine agonists such as alentemol, bromocriptine, fenoldopam, lisuride, naxagolide, pergolide and pratnipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

Deramciclane or a pharmaceutically acceptable salt thereof, may be administered in combination with a compound selected from the group consisting of: acetophenazine, alentemol, benzhexyl, bromocriptine, biperiden, chlorpromazine, chliorprotlhixene, clozapine, diazepain, fenoldopam, fluphenazine, haloperidol, levodopa, levodopa with benserazide, levodopa with carbidopa, lisuride, loxapine, inesoridazine, molindolone, naxagolide, olanzapine, pergolide, perphenazine, pimozide, pramipexole, risperidone, sulpiride, tetrabenazine, trihexyphenidyl, thioridazine, thiothixene and trifluoperazine.

Suitable neuroleptic agents of use in combination with deramcicline or a pharmaceutically acceptable salt thereof include the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with deramcicline may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

Creams, ointments, jellies, solutions, or suspensions containing the instant compounds can be employed for topical use. Mouth washes and gargles are included within the scope of topical use for the purposes of this invention.

A formulation intended for the oral administration to humans may conveniently contain from about 0.05 mg to about 5 g of active agent, compounded with an appropriate and convenient amount of carrier material which may vary from about 5 to about 95 percent of the total composition. Unit dosage forms can generally contain between from about 0.05 mg to about 1000 mg of the active ingredient.

The conditions recited herein can be treated or prevented by the administration of from about 0.01 mg to about 140 mg of the instant compounds per kilogram of body weight per day.

It is understood, however, that the specific dose level for any particular patient will depend upon a variety of factors. Such factors include the age, body weight, general health, sex, and diet of the patient. Other factors include the time and route of administration, rate of excretion drug combination, and the type and severity of the particular disease undergoing therapy. For example, inflammatory pain may be effectively treated by the administration of from about 0.01 mg to about 75 mg of the present compound per kilogram of body weight per day, or alternatively about 0.5 mg to about 3.5 g per patient per day. Neuropathic pain may be effectively treated by the administration of from about 0.01 mg to about 125 mg of the present compound per kilogram of body weight per day, or alternatively about 0.5 mg to about 5.5 g per patient per day.

The abbreviations used herein are as follows unless specified otherwise:
TEA Triethylamine
NaOAc Sodium Acetate
DMF Dimethylformamide
DIPEA Diisopropylethylamine
EtOAc Ethyl acetate
TFA Trifluoroacetic acid
TFAA Trifluoroacetic anhydride
THF Tetrahydrofuran
DMAP 4-Dimethylaminopyridine
RT Room temperature
H Hours
Min Minutes
DCM Dichloromethane
MeCN Acetonitrile
Selectfluor 1-(chloromethyl)-4-fluoro-1,4-diazoniabicyclo[2.2.2]octane ditetrafluoroborate
MeOH Methanol
EtOH Ethanol
IPA 2-Propanol
n-BuOH 1-Butanol
EDC 1-(3-Dimethylaminopropyl)-3-ethylcarbodiimide hydrochloride
HOBt 1-Hydroxy-benzotriazole
NMP N-Methyl-2-pyrrolidinone
DAST (Diethylamino)sulfur trifluoride

The compounds of the present invention can be prepared readily according to the following Schemes and specific examples, or modifications thereof, using readily available starting materials, reagents and conventional synthesis procedures. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following Schemes.

The preparation of compounds of Formula I can proceed through structures such as those of intermediate II.

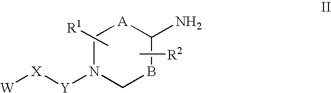

The synthesis of compounds of Formula I can be accomplished through intermediate II as depicted in Scheme 1. In general, intermediate II is alkylated with 4-chloro-1H-pyrazolo[3,4-d]pyrimidine 1 under standard alkylation conditions (R. K. Robins, 3. Amer. Chem. Soc. 78, 784-790 (1956)) such as hot alcoholic solvent, including isopropanol or 1-butanol, in the presence of base, including sodium carbonate or diisopropylethylamine. Intermediate II may also be alkylated with a suitably protected derivative of compound 1. Suitable protecting groups include alkoxymethyl derivatives such as N-tetrahydropyranyl, as illustrated by compound 2 and its isomer 3. Use of 2 and 3 in the alkylation reaction gives intermediates III and IV respectively. A suitable protecting group of this type is readily removed by treatment of intermediates III or IV with a protic acid, such as hydrochloric acid, and results in the formation of compounds of Formula I.

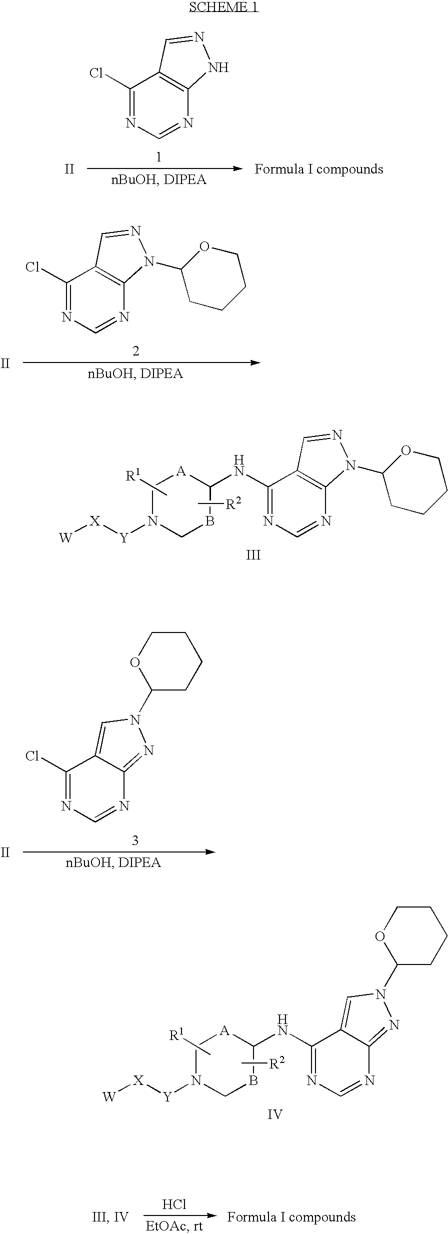

The synthesis of certain alkoxy-heterocycle containing compounds is depicted in Scheme 2. In the first step, suitably protected amine 4 is treated with halide 5 under basic conditions to give alkylated heterocycle 6. The nitrogen protecting group in 6 is removed under standard conditions such as acid hydrolysis and the intermediate 7 is converted to the final compound through the reaction sequence illustrated in Scheme 1. Alternatively, a suitably protected amino-heterocycle is alkylated with an epoxide (shown, for example, in Example 6), an aldehyde, or a ketone under reducing conditions (shown, for example, in Example 12); or the suitably protected amino-heterocycle is alkylated with an alpha-bromo ketone (shown, for example, in Example 14). In each of these cases, the intermediates can be further elaborated to final compounds using standard chemistry, including removal of the nitrogen protecting group, and through the reaction sequence illustrated in Scheme 1.

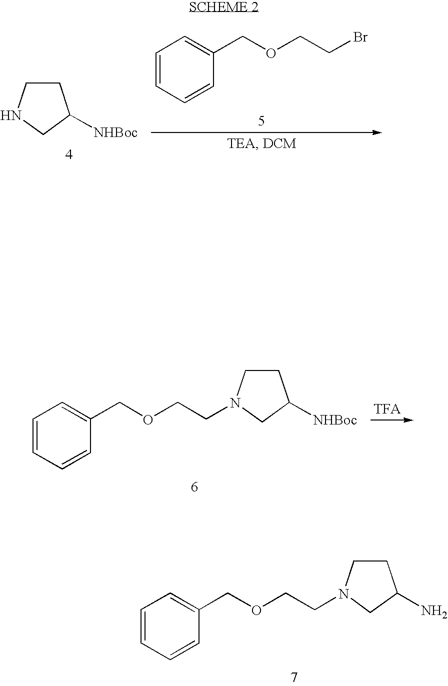

The synthesis of certain aryl-difluoro-ethyl containing compounds is depicted in Scheme 3. In the first step, arylation of 2-bromo-2,2-difluoroacetate 8 with 4-chloroiodobenzene 9 in the presence of copper bronze provides ester 10. An alternate synthesis of ester 10 involves acylation of an aryl ring with an acid chloride such as ethyl oxalylchloride in the presence of a Lewis acid such as aluminum trichloride, followed by halogenation of the ketoester with a reagent such as DAST. Hydrolysis of ester 10 under basic conditions provides acid 11. Carboxylic acid 11 is converted to acid chloride 12, which is used to acylate suitably protected amine 13 under standard conditions to yield amide 14. Amide 14 is deprotected under standard conditions such as acid hydrolysis to give amine 15. Amine 15 is reduced under standard conditions such as treatment with borane-THF complex or LAH to yield intermediate 16. Intermediate 16 is converted to the final compound through the reaction sequence illustrated in Scheme 1.

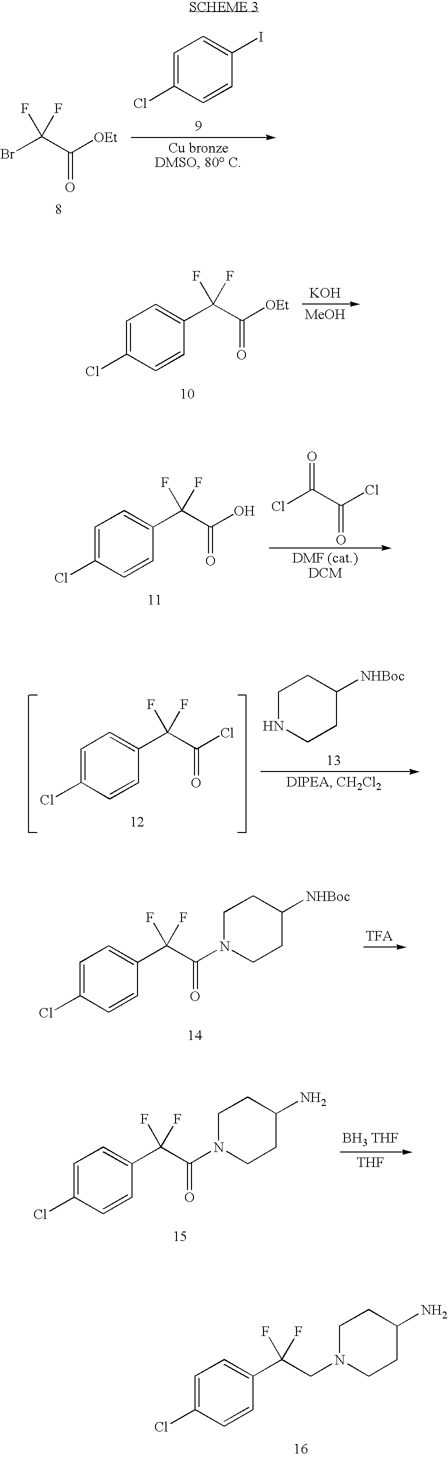

The synthesis of certain fluoropiperidine containing compounds is depicted in Scheme 4. In the first step ketone 17 is converted to silylenol-ether 18 by treatment with a silyl triflate under basic conditions. Treatment of 18 with an electrophilic fluorine source such as Selectfluor® (commercially available from Air Products and Chemicals, Inc., Allentown, Pa.) provides α-fluoroketone 19. Reduction of 19 under standard conditions provides alcohol 20. Alcohol 20 can be converted into a competent leaving group such as mesylate 21 and reacted with azide to give azide 22. Removal of the protecting group under standard conditions provides amine 23, which can be coupled with acid chloride 24 under standard conditions to provide amide 25. Reduction of amide 25 under standard conditions provides intermediate 26. Intermediate 26 is converted to the final compound through the reaction sequence illustrated in Scheme 1.

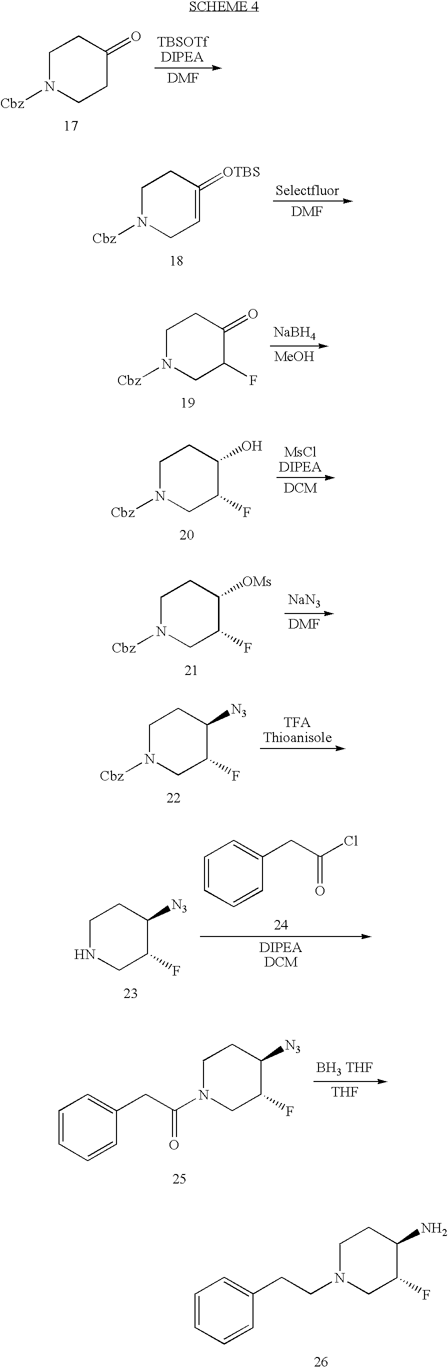

The synthesis of certain azabicyclo[3.1.0]hex-6-yl containing compounds can be achieved from the suitably protected diamine such as tert-butyl exo-3-azabicyclo[3.1.0]hex-6-ylcarbamate, which can be prepared according to literature procedures (Norris, T., Braish, T. F., Butters, M., DeVries, K. M., Hawkins, J. M., Massett, S. S., Rose, P. R., Santafianos, D., Sklavounos, C. J. Chem. Soc., Perkin Trans. 1 (2000) 1615-1622), through the reaction sequence illustrated in Schemes 1 and 3.

The synthesis of certain octahydrocyclopenta[c]pyrrol-4-yl containing compounds is depicted in Scheme 5. In the first step amine 27 is reacted with enone 28 tinder acidic conditions to give ketone 29. The ketone 29 can be reduced under standard conditions to give alcohol 30. Following a protecting group adjustment under standard conditions, the alcohol of 31 can be converted into a leaving group such as mesylate 32. Displacement of the mesylate by azide provides 33, and deprotection under standard conditions such as acid hydrolysis yields amine 34. Amine 34 is coupled with 24 under standard conditions to give amide 35. Reduction of amide 35 gives intermediate 36. Intermediate 36 is converted to the final compound through the reaction sequence illustrated in Scheme 1.

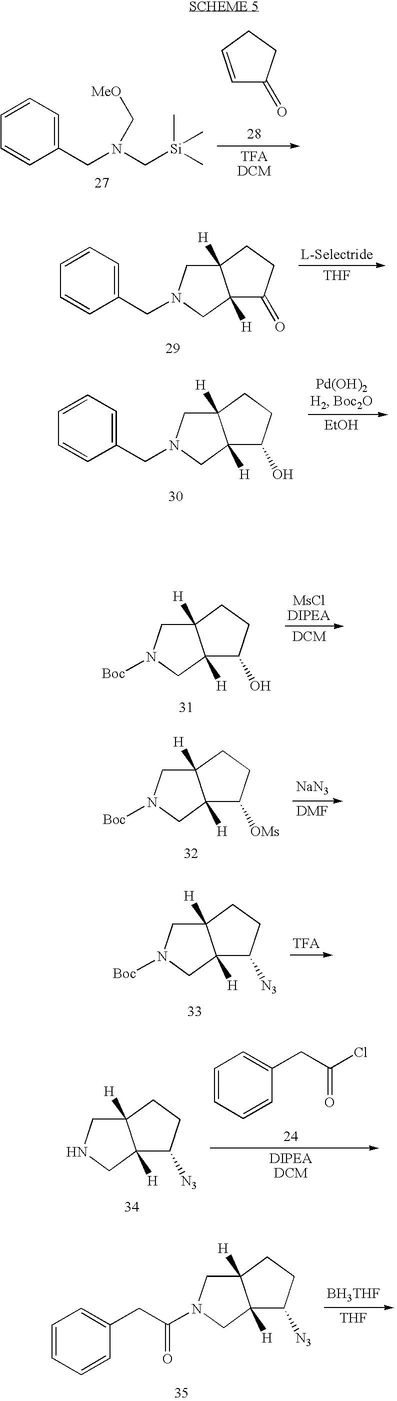

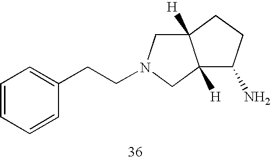

The synthesis of certain azabicyclo[3.2.1]oct-3-yl containing compounds is depicted in Scheme 6. In the first step ketone 37 is reacted with hydroxylamine hydrochloride to give imine 38. The h-nine 38 can be reduced under standard conditions to give endo 39. The related exo amine to 39 (exemplified by Example 45) is available by altering the reduction conditions to sodium metal in an alcoholic solvent such as propanol. Blocking the primary amine of endo 39 with a suitable protecting group provides 40 and deprotection of the secondary amine under suitable conditions such as acid hydrolysis yields amine 41. Amine 41 is coupled with 42 under standard conditions to give amide 43. Deprotection of amide 43 under standard conditions provides amide 44, and reduction of amide 44 gives intermediate 45. Intermediate 45 is converted to the final compound through the reaction sequence illustrated in Scheme 1.

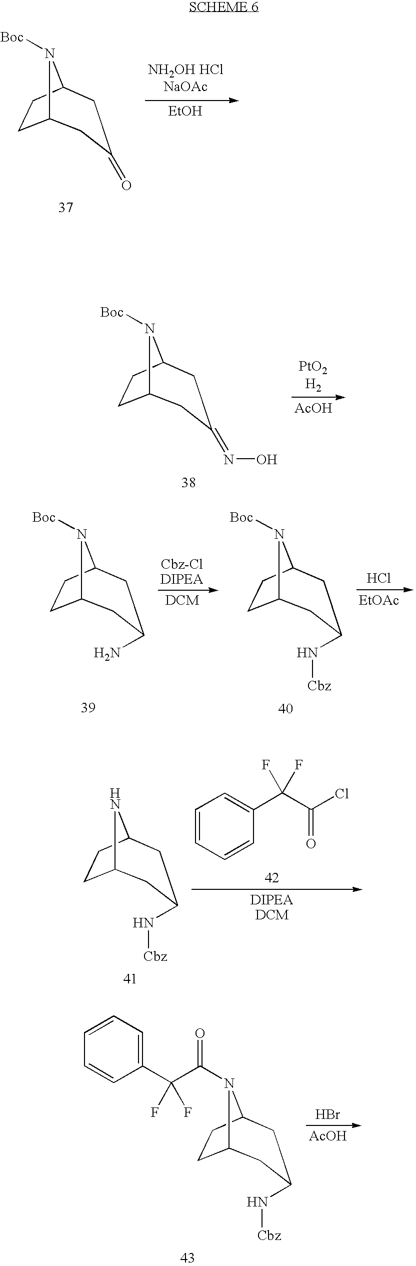

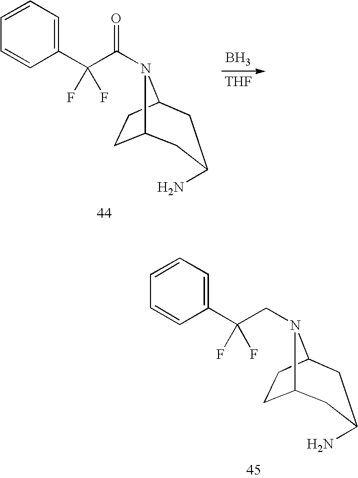

In some cases the final product may be further modified by, for example, manipulation of substituents. These manipulations may include, but are not limited to, reduction, oxidation, alkylation, acylation, halogenation and hydrolysis reactions which are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

### N-{1-[2-(benzyloxy)ethyl]pyrrolidin-3-yl}-1H-pyrazolo[3,4-d]pyrimidin-4-amine

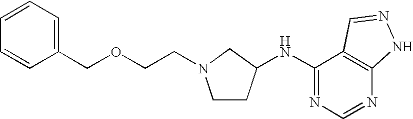

### Step A: tert-butyl {1-[2-(benzyloxy)ethyl]pyrrolidin-3-yl}carbamate

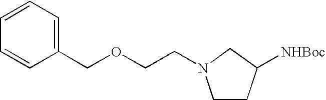

To a solution of tert-butyl pyrrolidin-3-ylcarbamate (1.5 g, 0.81 mmol) and TEA (2.3 mL 1.6 mmol) in methylene chloride (100 mL) was added [(2-bromoethoxy)methyl]benzene (1.7 g, 0.81 mmol) at room temperature. After 1 hour, 1M NaOH (500 mL) was added and the aqueous layer was extracted with methylene chloride. The combined organics were dried over sodium sulfate, filtered and concentrated. Purification by silica gel chromatography (1% isopropanol/methylene chloride->30% isopropanol/methylene chloride) gave the title compound (1.4 g). MS: 321 (M+H⁺).

### Step B: 1-[2-(benzyloxy)ethyl]pyrrolidin-3-amine

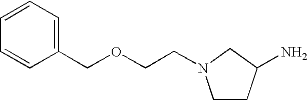

To tert-butyl {1-[2-(benzyloxy)ethyl]pyrrolidin-3-yl}carbamate (1.2 g, 3.9 mmol) was added trifluoroacetic acid (10 mL) and the resulting solution was stirred at room temperature. After 60 minutes, the reaction mixture was concentrated to give the trifluoroacetic acid salt of the title compound (1.2 g). MS: 221 (M+H⁺).

### Step C: N-{1-[2-(benzyloxy)ethyl]pyrrolidin-3-yl}-1H-pyrazolo[3,4-d]pyrimidin-4-amine

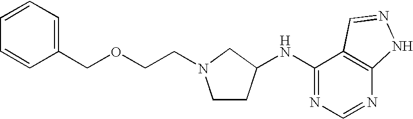

To a solution of 1-[2-benzyloxy)ethyl]pyrrolidin-3-amine (0.10 g, 0.35 mmol) in isopropanol (5 mL) was added DIPEA (2 mL) and 4-chloro-1-tetrahydro-pyran-2-yl)-1H-pyrazolo[3,4-d]pyrimidine (0.82 g, 0.35 mmol). The solution was then heated at 85° C. for 4 hours, after which it was cooled to room temperature and concentrated. The resulting residue was dissolved in methanol (3 mL) and treated with a saturated solution of HCl in ethyl acetate (10 mL). After 1 hour, the mixture was concentrated and the resulting residue was treated with saturated sodium bicarbonate and extracted with ethyl acetate. The combined organic layers were dried over sodium sulfate, filtered and concentrated to dryness. Purification by silica gel chromatography (2% isopropanol/methylene chloride→30% isopropanol/methylene chloride) gave the title compound (0.78 g) as a white solid. MS: 339 (M+H⁺); ¹H NMR (400 MHz, DMSO-d₆): δ 13.48 (s, 1H), 11.08-10.75 (m, 2H), 8.78 (s, 1H), 8.25 (s, 24), 7.32 (s, 5H), 4.78 (s, 1H), 4.48 (s, 1H), 3.98 (s, 1H), 3.78-3.22 (m, 4H), 2.48 (s, 2H), 2.09-1.94 (m, 2H), 1.81 (m, 1H).

The following Examples 2-5 were prepared using procedures similar to that described in Example 1 above.

### EXAMPLE 2

### N-{1-[2-(2-phenylethoxy)ethyl]pyrrolidin-3-yl}-1H-pyrazolo[3,4-d]pyrimidin-4-amine

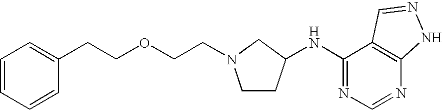

MS: 353 (M+H⁺); ¹H NMR (400 MHz, DMSO-d₆): δ 10.78 (s, 1H), 8.58 (s, 1H), 7.38-7.15 (m, 5H), 4.86 (s, 1H), 4.00 (m, 1H), 3.90 (m, 1H), 3.78-3.15 (m, 10H), 2.38-2.10 (m, 2H).

### EXAMPLE 3

### N-{1-[2-(benzyloxy)ethyl]piperidin-3-yl}-1H-pyrazolo[3,4-d]pyrimidin-4-amine

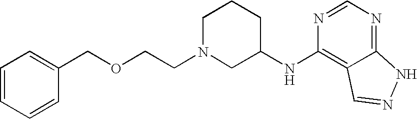

MS: 353 (M+H⁺); ¹H NMR (400 MHz, CD₃OD): δ 8.45 (s, 2H), 7.38-7.20 (m, 7H), 4.65 (s, 1H), 4.50-4.40 (m, 3H), 3.80-3.25 (m, 5H), 3.00-2.80 (m, 2H), 2.15-1.25 (m, 4H).

### EXAMPLE 4

### N-{1-[2-(benzyloxy)ethyl]piperidin-4-yl}-1H-pyrazolo[3,4-d]pyrimidin-4-amine

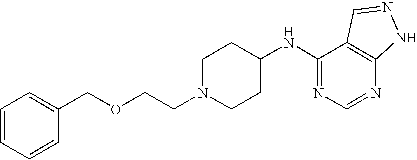

MS: 353 (M+H⁺); ¹H NMR (400 MHz, DMSO-d₆): δ 10.78 (s, 1H); 8.52 (s, 2H), 7.40-7.22 (m, 5H), 4.85 (s, 1H), 4.08 (m, 1H), 3.85 (m, 1H), 3.78-3.20 (m, 10H), 2.40-2.08 (m, 2H).

### EXAMPLE 5

### N-[1-(2-phenoxyethyl)piperidin-4-yl]-1H-pyrazolo[3,4-d]pyrimidin-4-amine

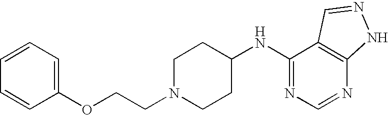

MS: 339 (M+H⁺); ¹H NMR (400 MHz, DMSO-d₆): δ 10.78 (s, 1H), 8.60 (s, 1H), 7.38-7.15 (m, 5H), 4.85 (s, 1H), 3.95 (m, 1H), 3.90 (m, 1H), 3.80-3.22 (m, 1H), 2.40-2.15 (m, 2H).

### EXAMPLE 6

### 1-phenyl-3-[3-(1H-pyrazolo[3,4-d]pyrimidin-4-ylamino)pyrrolidin-1-yl]propan-2-ol

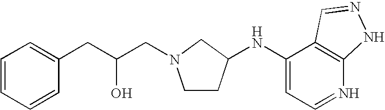

### Step A: tert-butyl[1-(2-hydroxy-3-phenylpropyl)pyrrolidin-3-yl]carbamate

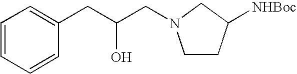

To a solution of tert-butyl pyrrolidin-3-ylcarbamate (3.1 g, 16 mmol) in ethanol (100 mL) was added 2-benzyloxirane (2.2 g, 16 mmol) and the resulting reaction mixture was heated at 10° C. After 1 hour, the reaction mixture was concentrated to give the title compound (2.5 g). MS: 321 (M+H⁺).

### Step B: 1-(3-aminopyrrolidin-1-yl)-3-phenylpropan-2-ol

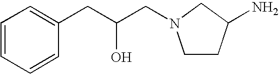

To tert-butyl[1-(2-hydroxy-3-phenylpropyl)pyrrolidin-3-yl]carbamate (1.5 g, 4.7 mmol) was added trifluoroacetic acid (0.75 mL) and the resulting solution was stirred at room temperature. After 30 minutes, the reaction mixture was concentrated to give the trifluoroacetic acid salt of the title compound (1.3 g). MS: 221 (M+H⁺).

### Step C: 1-phenyl-3-[3-(1H-pyrazolo[3,4-d]pyrimidin-4-ylamino)pyrrolidin-1-yl]propan-2-ol

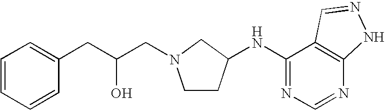

To a solution of 1-(3-aminopyrrolidin-1-yl)-3-phenylpropan-2-ol (0.22 g, 0.77 mmol) in 1-butanol (0.5 mL) was added DIPEA (0.5 mL) and 4-chloro-1-(tetrahydro-pyran-2-yl)-1H-pyrazolo[3,4-d]pyrimidine (0.18 g, 0.77 mmol) and the solution was heated at 150° C. for 10 minutes under microwave irradiation. The mixture was cooled and concentrated. The resulting residue was dissolved in methanol (3 mL) and treated with a saturated solution of HCl in ethyl acetate (10 mL). After 1 hour, the solution was concentrated and the residue was treated with saturated sodium bicarbonate and extracted with ethyl acetate. The combined organic layers were dried over sodium sulfate, filtered and concentrated to dryness. Purification by silica gel chromatography (1% isopropanol/methylene chloride→30% isopropanol/methylene chloride) gave the title compound (0.12 g) as a white solid. HRMS (M+H⁺): calculated=339.1928, observed=339.1918.

### EXAMPLE 7

### N-[1-(2,2-difluoro-3-phenylpropyl)pyrrolidin-3-yl]-1H-pyrazolo[3,4-d]pyrimidin-4-amine

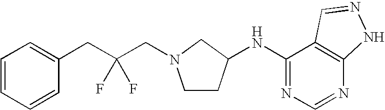

### Step A: tert-butyl[1-(2-oxo-3-phenylpropanoyl)pyrrolidin-3-yl]carbamate

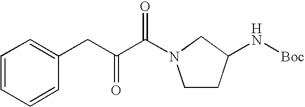

To a solution of tert-butyl pyrrolidin-3-ylcarbamate (4.4 g, 23 mmol) in methylene chloride (50 mL) was added 2-oxo-3-phenylpropanoic acid (3.8 g, 23 mmol), HOBT (3.2 g, 23 mmol), DIPEA (3.0 g, 23 mmol), and EDC (4.5 g, 23 mmol) at room temperature. After 1 hour; saturated sodium bicarbonate was added and the aqueous layer was extracted with ethyl acetate. The combined organics were dried over sodium sulfate, filtered and concentrated. Purification by silica gel chromatography (2% isopropanol/methylene chloride→30% isopropanol/methylene chloride) gave the title compound (2.5 g). MS: 333 (M+H⁺).

### Step B: tert-butyl[1-(2,2-difluoro-3-phenylpropanoyl)pyrrolidin-3-yl]carbamate

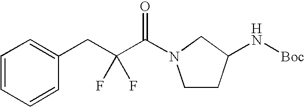

To a solution of tert-butyl[1-(2-oxo-3-phenylpropanoyl)pyrrolidin-3-yl]carbamate (2.5 g, 7.5 mmol) in methylene chloride (50 mL) was added DAST (2.4 g, 15 mmol) and the resulting solution was heated to 50° C. After 5 hour, the reaction mixture was cooled to room temperature, poured onto ice and extracted with ethyl acetate. The combined organics were dried over sodium sulfate, filtered and concentrated to give the title compound (2.2 g). MS: 335 (M+H⁺).

### Step C: 1-(2,2-difluoro-3-phenylpropyl)pyrrolidin-3-amine

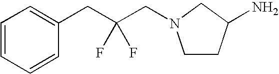

To tert-butyl[1-(2,2-difluoro-3-phenylpropanoyl)pyrrolidin-3-yl]carbamate (2.2 g, 6.4 mmol) was added trifluoroacetic acid (15 mL) and the resulting solution was stirred at room temperature. After 60 minutes, the reaction mixture was concentrated to dryness. The resulting residue was treated with 1M borane in THF (39 mL, 39 mmol) and heated to 80° C. for 1 hour. The reaction mixture was cooled to room temperature, quenched with 6N HCl (25 mL) and heated to 80° C. After 1 hour, the reaction mixture was cooled to room temperature, basified to pH>8 with NaOH and extracted with chloroform. The combined organics were dried over sodium sulfate, filtered and concentrated to give the title compound (1.7 g). MS: 241 (M+H⁺).

### Step D: N-[1-(2,2-difluoro-3-phenylpropyl)pyrrolidin-3-yl]-1H-pyrazolo[3,4-d]pyrimidin-4-amine

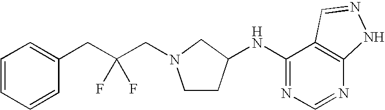

To a solution of 1-(2,2-difluoro-3-phenylpropyl)pyrrolidin-3-amine (0.070 g, 0.29 mmol) in 1-butanol (0.5 mL) was added DIPEA (0.5 mL) and 4-chloro-1-(tetrahydro-pyran-2-yl)-1H-pyrazolo[3,4-d]pyrimidine (0.070 g, 0.29 mmol) and the solution was heated at 150° C. for 10 minutes under microwave irradiation. The mixture was cooled and concentrated. The resulting residue was dissolved in methanol (3 mL) and treated with a saturated solution of HCl in ethyl acetate (10 mL). After 1 hour, the solution was concentrated and the residue was treated with saturated sodium bicarbonate and extracted with ethyl acetate. The combined organic layers were dried over sodium sulfate, filtered and concentrated to dryness. Purification by silica gel chromatography (2% isopropanol/methylene chloride→30% isopropanol/methylene chloride) gave the title compound (0.50 g) as a white solid. HRMS (M+H⁺): calculated 359.1751, observed=359.1804; ¹H NMR (400 MHz, CD₃OD): δ 8.30 (s, 1H), 8.10 (s, 1H), 7.30-7.20 (m, 5H), 4.75 (m, 1H), 3.28-3.20 (m, 5H), 3.08-2.90 (m, 2H), 2.80-2.62 (m, 4H), 2.40 (m, 1H), 11.90 (m, 1H).

### EXAMPLE 8

### N-[1-(2,2-difluoro-3-phenylpropyl)piperidin-4-yl]-1H-pyrazolo[3,4-d]pyrimidin-4-amine

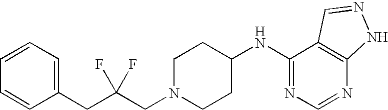

### Step A: Tert-butyl[1-(2-oxo-3-phenylpropanoyl)piperidin-4-yl]carbamate

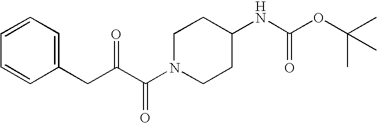

To a solution of 2-oxo-3-phenylpropanoic acid (0.53 g, 3.2 mmol) in methylene chloride (3.0 mL) was added oxalyl chloride (0.33 mL, 3.9 mmol) and a drop of DMF at room temperature. After 1 hour, DIPEA (2.5 mL, 14 mmol) was added followed by tert-butyl piperidin-4-ylcarbamate (0.71 g, 3.6 mmol). After 14 hours the reaction mixture was poured into 1M citric acid and extracted with ethyl acetate. The combined organic layers were washed with saturated sodium bicarbonate, water, brine, dried over sodium sulfate, filtered, and concentrated to give the title compound (0.95 g). MS: 291 (M-55).

### Step B: tert-butyl[1-(2,2-difluoro-3-phenylpropanoyl)piperidin-4-yl]carbamate

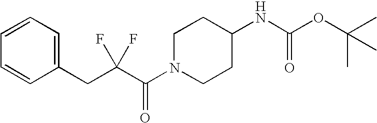

To a solution of tert-butyl[1-(2-oxo-3-phenylpropanoyl)piperidin-4-yl]carbamate (0.95 g, 2.7 mmol) in DCE (10 mL) was added DAST (0.68 mL, 5.5 mmol) at room temperature. The reaction mixture was heated to 60° C. After 14 hours, the reaction mixture was cooled to room temperature, poured into ice water and extracted with ethyl acetate. The combined organic layers were washed with 1M citric acid, 1M NaOH, brine, dried over sodium sulfate, filtered, and concentrated to give the title compound (0.94 g) as a brown solid. MS: 369 (M+H⁺).

### Step C: 1-(2,2-difluoro-3-phenylpropanoyl)piperidin-4-amine

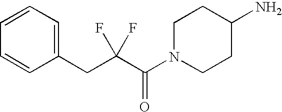

To a solution of tert-butyl[1-(2,2-difluoro-3-phenylpropanoyl)piperidin-4-yl]carbamate (0.94 g, 2.6 mmol) in methylene chloride (10 mL) was added a saturated solution of HCl in ethyl acetate (5.0 mL) at room temperature. After 2 hours, the reaction mixture was concentrated. The resulting residue was dissolved in acetonitrile/water (1:1), loaded onto an Mega BE-SCX ion exchange resin (commercially available from Varian Inc., Walnut Creek, Calif.), rinsed with acetonitrile, eluted with 10% ammonia in ethanol solution and concentrated. The resulting oil was dissolved in methylene chloride and methanol, treated with a saturated solution of HCl in ethyl acetate and concentrated to yield the hydrochloride salt of the title compound (0.10 g). MS: 269 (M+H⁺).

### Step D: 1-(2,2-difluoro-3-phenylpropyl)piperidin-4-amine

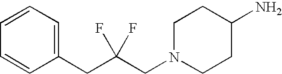

To a solution of 1-(2,2-difluoro-3-phenylpropanoyl)piperidin-4-amine (0.10 g, 0.30 mmol) in THF (1 mL) at 0° C. under nitrogen was added 1M borane in THF (3.6 mL, 3.6 mmol) and the reaction mixture was warmed to room temperature, and then heated to 70° C. After 2 hours, the reaction mixture was cooled to room temperature, quenched with 6N HCl (10 mL) and heated to 70° C. After 1 hour, the reaction mixture was basified to pH>8 with 5M NaOH and extracted with ethyl acetate. The combined organics were washed with water and brine, dried over sodium sulfate, filtered, and concentrated. The crude product was dissolved in methylene chloride and methanol, treated with a saturated solution of HCl in ethyl acetate and concentrated to yield the hydrochloride salt of the title compound (0.10 g). MS: 255 (M+H⁺).

### Step E: N-[1-(2,2-difluoro-3-phenylpropyl)piperidin-4-yl]-1H-pyrazolo[3,4-d]pyrimidin-4-amine

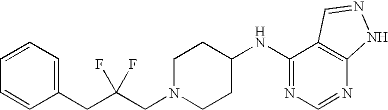

To a solution of 1-(2,2-difluoro-3-phenylpropyl)piperidin-4-amine (0.10 g, 0.31 mmol) in 1-butanol (2.5 mL) was added DIPEA (2.5 mL) and 4-chloro-1-(tetrahydro-pyran-2-yl)-1H-pyrazolo[3,4-d]pyrimidine (0.78 g, 0.33 mmol) and the solution was heated to 90° C. for 4 hours. The reaction mixture was cooled to room temperature and concentrated. The resulting residue was dissolved in methylene chloride and methanol, and treated with a saturated solution of HCl in ethyl acetate (5.0 mL) at room temperature for 2 hours. The reaction mixture was quenched with 1M NaOH and extracted with ethyl acetate. The combined organics were washed with brine, dried over sodium sulfate, filtered and concentrated. Purification by reverse phase chromatography (5% acetonitrile/0.1% trifluoroacetic acid/water→95% acetonitrile/0.1% trifluoroacetic acid/water, XTerra® MSC8 column available from Waters Corporation, Milford, Mass.) gave the product which was dissolved in methylene chloride and methanol, treated with a saturated solution of HCl in ethyl acetate and concentrated to yield the hydrochloride salt of the title compound (0.54 g) as a white solid. HRMS (M+H⁺): calculated=373.1947, observed=373.1964; ¹H NMR (400 MHz, CD₃OD): δ 8.60-8.52 (s, 2H), 7.41-7.31 (m, 5H), 4.69-4.51 (s, 1H), 3.86-3.68 (m, 4H), 3.46-3.33 (m, 4H), 2.41-2.31 (m, 2H), 2.19-2.03 (m, 2H).

### EXAMPLE 9

### N-[1-(2-phenylethyl)piperidin-4-yl]-1H-pyrazolo[3,4-d]pyrimidin-4-amine

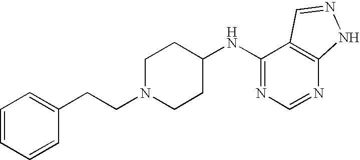

The title compound was prepared using procedures similar to that reported in Example 1 above. HRMS (M+H⁺): calculated=323.1373, observed=323.1999; ¹H NMR (500 MHz, CD₃OD): δ 8.68-5.85 (m, 2H), 7.40-7.25 (m, 5H), 4.75-4.65 (m, 1H), 3.86-3.79 (m, 2H), 3.44-3.38 (m, 1H), 3.17-3.10 (m 2H), 2.47-2.39 (m, 2H), 2.18-2.07 (m, 2H).

### EXAMPLE 10

### N-[1-(3-phenylpropyl)piperidin-4-yl]-1H-pyrazolo[3,4-d]pyrimidin-4-amine

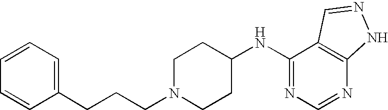

The title compound was prepared using procedures similar to that reported in Example 1 above. HRMS (M+H⁺): calculated=337.2135, observed=337.2164; ¹H NMR (500 MHz, CD₃OD): δ 8.23-8.19 (s, 1H), 8.15-8.11 (s, 1H), 7.29-7.23 (m, 2H), 7.23-7.18 (m, 2H), 7.18-7.13 (m, 1H), 4.19-4.09 (m, 1H), 3.06-2.98 (m, 2H), 2.68-2.61 (m, 2H), 2.47-2.39 (m, 2H), 2.24-2.13 (m, 2H), 2.10-2.01 (m, 2H), 1.91-1.82 (m, 2H), 1.75-1.64 (m, 2H).

### EXAMPLE 11

### N-[(1S,3R)-3-(3-phenyl-1,2,4-oxadiazol-5-yl)cyclopentyl]-1H-pyrazolo[3,4-d]pyrimidin-4-amine

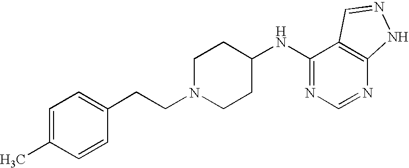

### Step A: tert-butyl {1-[2-(4-methylphenyl)ethyl]piperidin-4-yl}carbamate

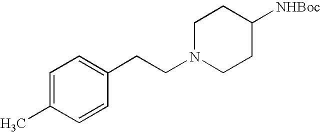

To a solution of tert-butyl piperidin-4-ylcarbamate (0.40 g, 2.0 mmol) in toluene (1.0 mL) was added 2-(4-methylphenyl)ethanol (0.27 g, 2.0 mmol), potassium carbonate (0.014 g, 0.10 mmol) and dichloro(pentamethylcyclopentadienyl)iridium (III) dimer (0.040 g, 0.050 mmol) under an atmosphere of nitrogen, and the reaction mixture was heated to 110° C. After 17 hours the reaction mixture filtered, rinsed with ethyl acetate and concentrated. Purification by silica gel chromatography (1% isopropanol/methylene chloride→40% isopropanol/methylene chloride) gave the title compound (0.25 g) as a solid. HRMS (M+H⁺): calculated=319.2380, observed=319.2373.

### Step B: N-[(1S,3R)-3-(3-phenyl-1,2,4-oxadiazol-5-yl)cyclopentyl]-1H-pyrazolo[3,4-d]pyrimidin-4-amine

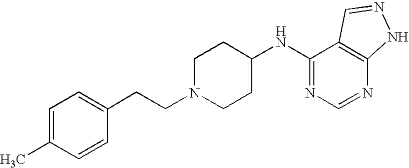

The title compound was prepared using procedures similar to that reported in Example 8, Steps C and E above. HRMS (M+H⁺): calculated=337.2135, observed=337.2145; ¹H NMR (400 z, CD₃OD): δ 8.22 (s, 1H), 8.13 (s, 1H), 7.10 (s, 4H), 4.17 (s, 1H), 3.13-3.10 (m, 2H), 2.82-2.78 (m, 2H), 2.65-2.61 (m, 2H), 2.29 (s, 1H), 2.11-2.08 (m, 2H), 1.77-1.72 (m, 2H).

### EXAMPLE 12

### N-{1-[1-methyl-2-(4-methylphenyl)ethyl]piperidin-4-yl}-1H-pyrazolo[3,4-d]pyrimidin-4-amine

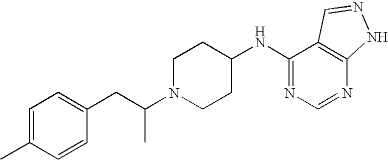

### Step A: Tert-butyl {1-[1-methyl-2-(4-methylphenyl)ethyl]piperidin-4-yl}carbamate

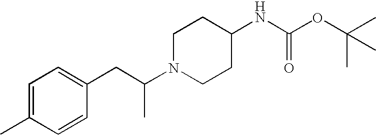

To a neat mixture of tert-butyl piperidin-4-ylcarbamate (0.85 g, 4.2 mmol) and 1-(4-methylphenyl)acetone (0.55 mL, 3.5 mmol) was added titanium isopropoxide (2.1 mL, 7.1 mmol) at room temperature under nitrogen. After 20 hours, a solution of sodium cyanoborohydride (0.44 g, 7.1 mmol) in EtOH (10 mL) was added at room temperature. After an additional 16 hours, the reaction mixture was slowly quenched by the addition of 1M NaOH, and the resulting precipitate was filtered through celite and rinsed with ethyl acetate. The filtrate was washed with water, brine, dried over sodium sulfate, filtered, and concentrated. Purification by silica gel chromatography (1% isopropanol/methylene chloride→20% isopropanol/methylene chloride) gave the title compound (0.86 g) as a yellow oil. HRMS (M+H⁺): calculated=333.2537, observed=333.2527.

### Step B: N-{1-[1-methyl-2-(4-methylphenyl)ethyl]piperidin-4-yl}-1H-pyrazolo[3,4-d]pyrimidin-4-amine

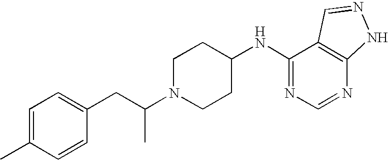

The title compound was prepared using procedures similar to that reported in Example 8, Steps C and E above. HRMS (M+H⁺): calculated=351.2292, observed=351.2291; ¹H NMR (500 MHz, CD₃OD): δ 8.71-8.65 (s, 1H), 8.63-8.56 (s, 1H), 7.24-7.11 (m, 4H), 4.78-4.65 (m, 1H), 3.82-3.52 (m, 3H), 3.48-3.36 (m, 2H), 3.36-3.33 (m, 1H), 2.80-2.69 (m, 1H), 2.49-2.36 (s, 2H), 2.35-2.29 (s, 3H), 2.29-2.16 (m 2H), 1.33-1.21 (m, 3H).

### EXAMPLE 13

### N-(1-benzylpiperidin-4-yl)-1H-pyrazolo[3,4-d]pyrimidin-4-amine

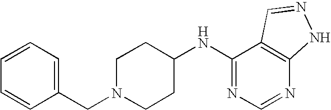

The title compound was prepared using procedures similar to that reported in Example 1 above. HRMS (M+H⁺): calculated=309.1822, observed=309.1839; ¹H NMR (500 MHz, CD₃OD): δ 8.24-8.18 (s, 1H), 8.16-8.09 (s, 1H), 7.39-7.31 (m, 4H), 7.31-7.25 (m, 1H), 4.19-4.08 (m, 1H), 3.60-3.55 (s, 2H), 3.02-2.94 (m, 2H), 2.28-2.17 (m, 2H), 2.08-2.00 (m, 2H), 1.76-1.65 (m, 2H).

### EXAMPLE 14

### 1-phenyl-2-[4-(1H-pyrazolo[3,4-d]pyrimidin-4-ylamino)piperidin-1-yl]ethanol

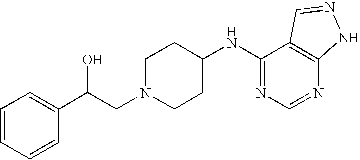

### Step A: tert-butyl[1-(2-oxo-2-phenylethyl)piperidin-4-yl]carbamate

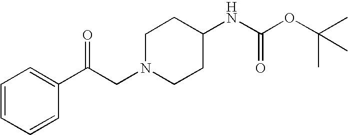

To a solution of 2-bromo-1-phenylethanone (0.63 g, 3.2 mmol) and tert-butyl piperidin-4-ylcarbamate (0.68 g, 3.4 mmol) in THF (5 mL) was added DIPEA (0.57 mL, 3.5 mmol) and the mixture was heated to 130° C. for 10 minutes under microwave irradiation. The reaction mixture was poured into 1M NaOH and extracted with ethyl acetate. The combined organics were washed with brine, dried over sodium sulfate, filtered, and concentrated. Purification by silica gel chromatography (50% ethyl acetate/hexane→100% ethyl acetate/hexane) gave the title compound (0.79 g) as a yellow solid. HRMS (M+H⁺): calculated=319.2016, observed=319.2022.

### Step B: 2-(4-aminopiperidin-1-yl)-1-phenylethanone

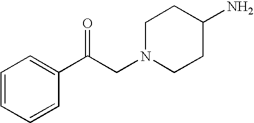

The title compound was prepared from tert-butyl[1-(2-oxo-2-phenylethyl)piperidin-4-yl]carbamate according to the procedure reported in Example 8, Step C presented above. MS: 219 (M+H⁺).

### Step C: 2-(4-aminopiperidin-1-yl)-1-phenylethanol

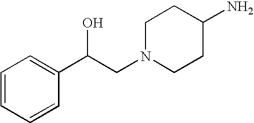

To a solution of 2-(4-aminopiperidin-1-yl)-1-phenylethanone (0.35 g, 1.1 mmol) in MeOH (3 mL) was added sodium borohydride (0.13 g, 3.3 mmol) at room temperature. After 1 hour, the reaction mixture was quenched by dropwise addition of water (3 mL). The reaction mixture was poured into 1M NaOH and extracted with ethyl acetate. The combined organics were washed with brine, dried over sodium sulfated, filtered, and concentrated to yield the title compound (0.34 g) as a white solid. HRMS (M+H⁺): calculated=221.1649, observed=221.1657.

### Step D: 1-phenyl-2-[4-(1H-pyrazolo[3,4-d]pyrimidin-4-ylamino)piperidin-1-yl]ethanol

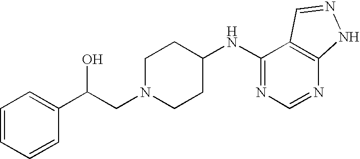

The title compound was prepared using procedures similar to that reported in Example 1, Step C above. HRMS (M+H⁺): calculated=339.1928, observed=339.1935; ¹H NMR (400 MHz, CD₃OD): δ 8.68-8.63 (m, 1H), 8.63-8.55 (m, 1H), 7.53-7.45 (m, 2H), 7.45-7.30 (m, 3H), 5.22-5.12 (m, 1H), 4.76-4.61 (m, 1H), 4.02-3.91 (m, 1H), 3.89-3.79 (m, 1H), 3.76-3.45 (m, 1H), 2.52-2.28 (m, 2H), 2.28-2.04 (m, 2H).

### EXAMPLE 15

### 1-phenyl-2-[4-(1H-pyrazolo[3,4-d]pyrimidin-4-ylamino)piperidin-1-yl]ethanone

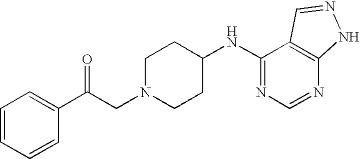

The title compound was prepared from 2-(4-aminopiperidin-1-yl)-1-phenylethanone (Example 14, Step B) according to the procedure reported in Example 1, Step C above. HRMS (M+H⁺): calculated=337.1772, observed=337.1778; ¹H NMR (500 MHz, CD₃OD): δ 8.25 (s, 1H), 8.17 (s, 1H), 8.06-8.00 (m, 2H), 7.65-7.59 (m, 1H), 7.54-7.47 (m, 2H), 4.24-4.14 (m, 1H), 4.02 (s, 2H), 3.16-3.07 (m, 2H), 2.44-2.34 (m, 2H), 2.11-2.03 (m, 2H), 1.86-1.75 (m, 21-1).

### EXAMPLE 16

### (1R)-1-(2-fluorophenyl)-2-[4-1H-pyrazolo[3,4-d]pyrimidin-4-ylamino)piperidin-1-yl]ethanol

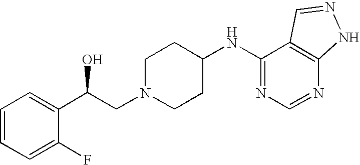

The title compound was prepared from (2R)-2-fluorophenyloxirane according to the procedure reported in Example 6 presented above. MS 357 (M+H⁺).

### EXAMPLE 17

### N-[1-(2-fluoro-2-phenylethyl)piperidin-4-yl]-1H-pyrazolo[3,4-d]pyrimidin-4-amine

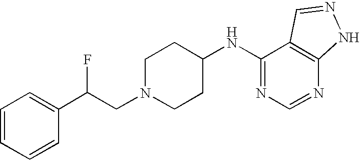

### Step A: Methyl fluoro(phenyl)acetate

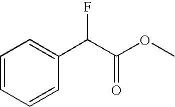

To a solution of methyl hydroxy(phenyl)acetate (2.0 g, 12 mmol) in methylene chloride (25 mL) under nitrogen was added DAST (2.5 mL, 20 mmol) at 0° C. and the reaction mixture was allowed to warm to room temperature. After 1 hour, the reaction mixture was poured into ice water and extracted with ethyl acetate. The combined organics were washed with brine, dried over sodium sulfate, filtered, and concentrated to give the title compound (2.0 g) as a yellow oil. MS: 337 (2M+H⁺).

### Step B: Fluoro(phenyl)acetic Acid

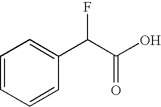

To a solution of methyl fluoro(phenyl)acetate (2.0 g, 12.0 mmol) in methanol (20 mL) and water (5 mL) was added ground KOH pellets (2.0 g, 36 mmol) at 0° C. and the reaction mixture was permitted to warm to room temperature. After 3 hours, the reaction mixture was cooled to 0° C., acidified to pH<3 with 6N HCl and extracted with ethyl acetate. The combined organics were washed with brine, dried over sodium sulfate, filtered, and concentrated to yield the title compound (1.8 g) as a brown solid.

### Step C: N-[1-(2-fluoro-2-phenylethyl)piperidin-4-yl]-1H-pyrazolo[3,4-d]pyrimidin-4-amine

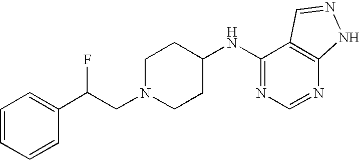

The title compound was prepared using procedures similar to that reported in Example 8, Steps A, C, D and E above. HRMS (M+H⁺): calculated=341.1885, observed=341.1888; ¹H NMR (400 MHz, CD₃OD): δ 8.70-8.65 (s, 1H), 8.64-8.59 (s, 1H), 7.55-7.43 (m, 5H), 6.25-6.18 (m, 0.5H), 6.13-6.06 (m, 0.5H), 4.79-4.64 (m, 1H), 4.03-3.95 (m, 1H), 3.95-3.70 (m, 2H), 3.69-3.47 (m, 1H), 3.47-3.33 (m, 2H), 2.52-2.32 (m, 2H), 2.30-2.10 (m, 2H).

### EXAMPLE 18

### N-[1-(2,2-difluoro-2-phenylethyl)piperidin-4-yl]-1H-pyrazolo[3,4-d]pyrimidin-4-amine

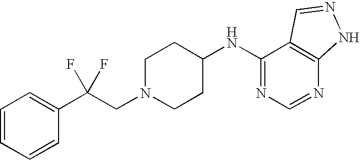

The title compound was prepared from difluoro(phenyl)acetic acid (Hagele, G., Haas, A. J. of Fluorine Chemistry (1996) 76, 15-19) according to the procedure reported in Example 8, Steps A, C, D and E presented above. HRMS (M+H⁺): calculated=359.1791, observed=359.1797; ¹H NMR (400 MHz, CD₃OD): δ 8.82-8.72 (s, 1H), 8.64-8.58 (s, 1H), 7.70-7.63 (m, 2H), 7.63-7.53 (m, 3H), 4.78-4.64 (m, 1H), 4.24-4.08 (m, 2H), 4.01-3.87 (m, 2H), 3.63-3.47 (m, 2H), 2.49-2.37 (m, 2H), 2.35-2.20 (m, 2H).

### EXAMPLE 19

### N-{1-[2-(4-chlorophenyl)-2,2-difluoroethyl]piperidin-4-yl}-1H-pyrazolo[3,4-d]pyrimidin-4-amine

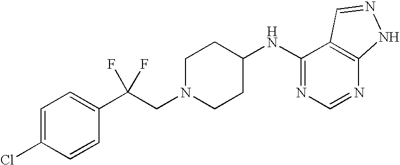

### Step A: (4-chlorophenyl)(difluoro)acetic acid

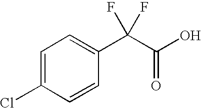

To a solution of 1-chloro-4-iodobenzene (5.9 g, 25 mmol) in DMSO (100 mL) was added ethyl bromo(difluoro)acetate (5.0 g, 25 mmol) and copper powder (3.1 g, 49 mmol) and the mixture was heated at 80° C. After 20 hours, the reaction mixture was poured into a solution of dibasic potassium hydrogen phosphate, trihydrate (56 g, 250 mmol) in water (500 mL) with vigorous stirring. The suspension was filtered and the solid was rinsed with ether. The filtrate was added to brine and extracted with ether (2×). The combined organics were washed with brine, dried over sodium sulfate, filtered, and concentrated. The resulting oil was dissolved in methanol (100 mL) and treated with 50% aqueous KOH (250 mL) at room temperature. After 2 hours, the reaction mixture was concentrated, dissolved in CHCl₃ and extracted with 1M HCl. The aqueous layer was basified with NaOH and extracted with CHCl₃. The combined organics were dried over sodium sulfate, filtered, and concentrated to yield the title compound (3.5 g) as a waxy solid.

### Step B: Tert-butyl {1-[(4-chlorophenyl)(difluoro)acetyl]piperidin-4-yl}carbamate

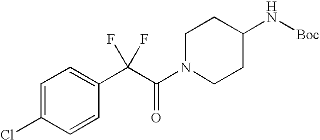

The title compound was prepared using procedures similar to that reported in Example 8, Step A above. MS: 389 (M+H⁺).

### Step C: 1-[2-(4-chlorophenyl)-2,2-difluoroethyl]piperidin-4-amine

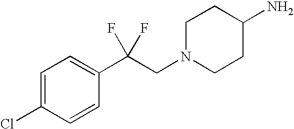

To tert-butyl {1-[(4-chlorophenyl)(difluoro)acetyl]piperidin-4-yl} (0.42 g, 1.1 mmol) was added trifluoroacetic acid (5 mL) and the resulting solution was stirred at room temperature. After 60 minutes, the reaction mixture was concentrated, treated with a saturated solution of HCl in ethyl acetate and concentrated. To the resulting residue under nitrogen was added THF (1 mL) and 1M borane in THF (6.6 mL, 6.6 mmol) and the reaction mixture was heated to 70° C. After 2 hours, the reaction mixture was cooled to room temperature, quenched with 6N HCl (20 mL) and heated to 70° C. After 1 hour, the reaction mixture was basified to pH>8 with 5M NaOH and extracted with ethyl acetate. The combined organics were washed with water and brine, dried over sodium sulfate, filtered, and concentrated to give the title compound (0.25 g). MS: 275 (M+H⁺).

### Step D: N-{1-[2-(4-chlorophenyl)-2,2-difluoroethyl]piperidin-4-yl}-1H-pyrazolo[3,4-d]pyrimidin-4-amine

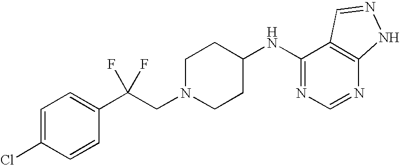

The title compound was prepared using procedures similar to that reported in Example 8, Step E above. HRMS (M+H⁺): calculated 393.1401, observed 393.1421; ¹H NMR (400 MHz, CD₃OD): δ 8.20 (s, 1H), 8.10 (s, 1H), 7.58-7.42 (m, 4H), 4.10 (m, 1H), 3.10-2.95 (m, 2H), 2.86 (m, 2H), 2.48 (m, 2H), 1.85 (m, 2H), 1.58 (m, 2H).

### EXAMPLE 20

### N-{1-[2,2-difluoro-2-(4-methylphenyl)ethyl]piperidin-4-yl}-1H-pyrazolo[3,4-d]pyrimidin-4-amine

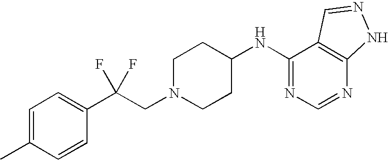

The title compound was prepared using procedures similar to that reported in Example 19 above. HRMS (M+H⁺): calculated=373.1947, observed=373.1961; ¹H NMR (500 MHz, CD₃OD): δ 8.75-8.63 (s, 1H), 8.63-8.57 (s, 1H), 7.56-7.48 (m, 2H), 7.41-7.34 (m, 2H), 4.76-4.62 (m, 1H), 4.17-4.01 (m, 2H), 3.99-3.83 (m, 2H), 3.59-3.41 (m, 2H), 2.47-2.36 (m, 5H), 2.31-2.13 (m, 2H).

### EXAMPLE 21

### N-{1-[2,2-difluoro-2-(4-fluorophenyl)ethyl]piperidin-4-yl}-1H-pyrazolo[3,4-d]pyrimidin-4-amine

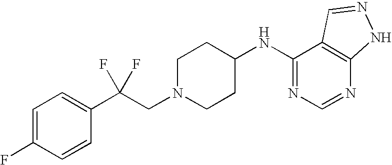

The title compound was prepared from difluoro(4-fluorophenyl)acetic acid (Hagele, G., Haas, A. J. of Fluorine Chemistry (1996) 76, 15-19) using procedures similar to that reported in Example 19, Steps B, C and D above. HRMS (M+H⁺): calculated=377.1696, observed=377.1690; ¹H NMR (400 MHz, CD₃OD): δ 8.68-8.57 (m, 2H), 7.74-7.66 (m, 2H), 7.36-7.29 (m, 2H), 4.73-4.62 (m, 1H), 4.17-4.03 (m, 2H), 3.95-3.82 (m, 2H), 3.54-3.41 (m, 2H), 2.47-2.37 (m, 2H), 2.26-2.12 (m, 2H).

### EXAMPLE 22

### N-{1-[2,2-difluoro-2-(2-fluorophenyl)ethyl]piperidin-4-yl}-1H-pyrazolo[3,4-d]pyrimidin-4-amine

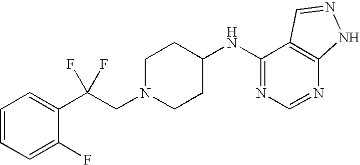

The title compound was prepared using procedures similar to that reported in Example 19 above. HRMS (M+H⁺): calculated=377.1696, observed=377.1677; ¹H NMR (400 MHz, CD₃OD): δ 8.71-8.63 (s, 1H), 8.63-8.57 (s, 1H), 7.73-7.61 (m, 2H), 7.44-7.30 (m, 2H), 4.75-4.63 (m, 1H), 4.24-4.09 (m, 2H), 3.96-3.82 (m, 2H), 3.59-3.43 (m, 2H), 2.46-2.36 (m, 2H), 2.27-2.13 (m, 2H).

### EXAMPLE 23

### N-{1-[2-(2,6-difluorophenyl)-2,2-difluoroethyl]piperidin-4-yl}-1H-pyrazolo[3,4-d]pyrimidin-4-amine

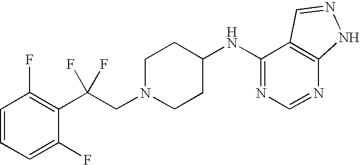

### Step A: 1,3-difluoro-2-iodobenzene

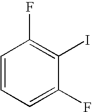

To a solution of 2-bromo-1,3-difluorobenzene (5.7 g, 30 mmol) in 1,4-dioxane was added copper iodide (0.28 g, 1.5 mmol), sodium iodide (8.9 g, 59 mmol) and N,N-dimethylethane-1,2-diamine (0.32 mL, 3.0 mmol) and the reaction mixture was heated at 110° C. under an atmosphere of nitrogen. After 48 hours, the reaction mixture was poured into an aqueous solution of ammonium hydroxide (20 mL in 200 mL water) and extracted with ethyl acetate. The combined organics were washed with brine, dried over sodium sulfate, filtered, & concentrated to yield the title compound (5.1 g).

### Step B: N-{1-[2-(2,6-difluorophenyl)-2,2-difluoroethyl]piperidin-4-yl}-1H-pyrazolo[3,4-d]pyrimidin-4-amine

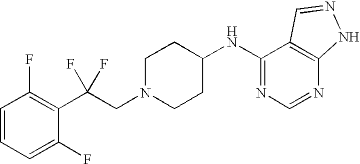

The title compound was prepared according to the procedure reported in Example 19 presented above. HRMS (M+H⁺): calculated=395.1602, observed=395.1598; ¹H NMR (400 MHz, CD₃OD): δ 8.65-8.61 (s, 1H), 8.61-8.58 (s, 1H), 7.71-7.62 (m, 1H), 7.23-7.15 (m, 2H), 4.70-4.59 (m, 1H), 4.23-4.06 (m, 2H), 3.88-3.76 (m, 2H), 3.50-3.38 (m, 2H), 2.43-2.34 (m, 2H), 2.21-2.07 (m, 2H).

### EXAMPLE 24

### N-(1-{2,2-difluoro-2-[4-(trifluoromethyl)phenyl]ethyl}piperidin-4-yl)-1H-pyrazolo[3,4-d]pyrimidin-4-amine

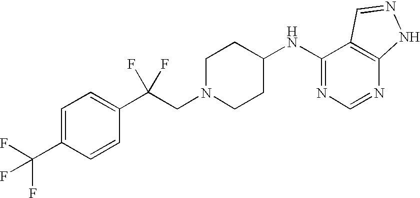

Title compound was prepared using procedures similar to that reported in Example 19 above. HRMS (M+H⁺): calculated=427.1664, observed=427.1645; ¹H NMR (400 MHz, CD₃OD): δ 8.73-8.65 (s, 1H), 8.64-8.57 (s, 1H), 7.94-7.84 (m, 4H), 4.76-4.63 (m, 1H), 4.22-4.08 (m, 2H), 3.96-3.84 (m, 2H), 3.56-3.43 (m, 2H), 2.47-2.37 (m, 2H), 2.29-2.15 (m, 2H).

### EXAMPLE 25

### N-{1-[2-(4-bromophenyl)-2,2-difluoroethyl]piperidin-4-yl}-1H-pyrazolo[3,4-d]pyrimidin-4-amine

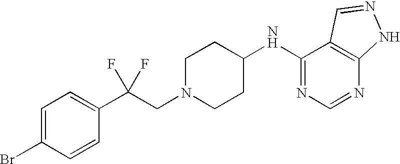

### Step A: difluoro(4-bromophenyl)acetic acid

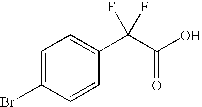

To a suspension of aluminum trichloride (2.2 g, 16 mmol) and ethyl chloro(oxo)acetate (1.9 mL, 16 mmol), was added bromobenzene (1.5 mL, 14 mmol) dropwise at room temperature under an atmosphere of nitrogen. After 24 hours a solution of ice water and saturated sodium bicarbonate solution was added dropwise. The aqueous layer was extracted with diethyl ether and the combined organics were washed with brine, dried over sodium sulfate, filtered, and concentrated. Purification by silica gel chromatography (1% ethyl acetate/hexane→10% ethyl acetate/hexane) gave the keto-ester (1.4 g) as a yellow oil. To the resulting oil (1.4 g, 5.6 mmol) in DCE (15 mL) was added DAST (2.1 mL, 17 mmol) at room temperature. The reaction mixture was heated to 60° C. After 14 hours, the reaction mixture was cooled to room temperature, poured into ice water and extracted with diethyl ether. The combined organiclayers were washed with brine, dried over sodium sulfate, filtered, and concentrated to give the difluoro ester as an oil. The resulting oil was dissolved in methanol:water (100 mL: 20 mL), cooled to 0° C. and added KOH pellets (1.0 g, 17 mmol) while stirring. After 2 hours, the reaction mixture was washed with methylene chloride. Combined aqueous layers were then basified to pH>8 with 5M NaOH & extracted with diethyl ether. The combined organics were washed with brine, dried over sodium sulfate, filtered, and concentrated to yield the title compound (1.4 g) as a waxy solid.

### Step B: N-{1-[2-(4-bromophenyl)-2,2-difluoroethyl]piperidin-4-yl}-1H-pyrazolo[3,4-a]pyrimidin-4-amine

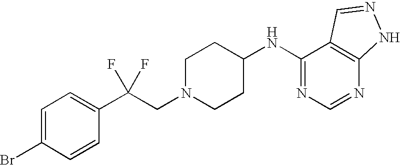

The title compound was prepared from difluoro(4-bromophenyl)acetic acid using procedures similar to that reported in Example 19, Steps B, C, and D above. HRMS (M+H⁺): calculated=437.0876, observed=437.0846; ¹H NMR (500 MHz, CD₃OD): δ 8.72-8.66 (s, 1H), 8.63-8.60 (s, 1H), 7.79-7.74 (m, 2H), 7.61-7.56 (m, 2H), 4.75-4.65 (m, 1H), 4.17-4.07 (m, 2H), 3.95-3.87 (m, 2H), 3.55-3.43 (m, 2H), 2.47-2.38 (m, 2H), 2.28-2.16 (m, 2H).

### EXAMPLE 26

### N-{1-[2-(4-cyclopropylphenyl)-2,2-difluoroethyl]piperidin-4-yl}-1H-pyrazolo[3,4-d]pyrimidin-4-amine

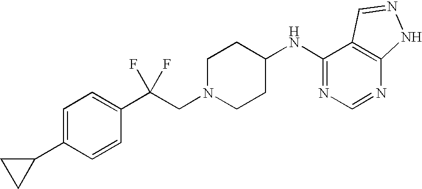

The title compound was prepared using procedures similar to that reported in Example 25 above. HRMS (M+H⁺): calculated=399.2104, observed=399.2110; ¹H NMR (500 MHz, CD₃OD): δ 8.70-8.64 (s, 1H), 8.64-8.58 (s, 1H), 7.54-7.48 (m, 2H), 7.29-7.23 (m, 2H), 4.76-4.63 (m, 1H), 4.14-4.01 (m, 2H), 3.97-3.85 (m, 2H), 3.56-3.41 (m, 2H), 2.47-2.37 (m, 2H), 2.27-2.14 (m, 2H), 2.04-1.96 (m, 1H), 1.09-1.03 (m, 2H), 0.79-0.73 (m, 2H).

### EXAMPLE 27

### N-{1-[2,2-difluoro-2-(4-methoxyphenyl)ethyl]piperidin-4-yl}-1H-pyrazolo[3,4-d]pyrimidin-4-amine

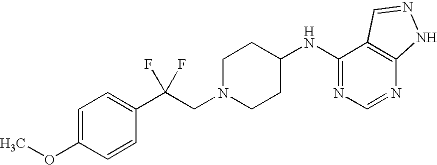

The title compound was prepared using procedures similar to that reported in Example 25 above. HRMS (M+H⁺): calculated=389.1896, observed=389.1905; 114 NMR (500 MHz, CD₃OD): b 8.22-8.18 (s, 1H), 8.14-8.07 (s, 1H), 7.49-7.44 (m, 2H), 7.01-6.96 (m, 2H), 4.13-4.02 (m, 1H), 3.85-3.82 (s, 3H), 3.06-2.98 (m, 2H), 2.94-2.88 (m, 2H), 2.49-2.40 (m, 2H), 1.98-1.90 (m, 2H), 1.68-1.57 (m, 2H).

### EXAMPLE 28

### N-[(3R,4R)-3-fluoro-1-(2-phenylethyl)piperidin-4-yl]-1H-pyrazolo[3,4-d]pyrimidin-4-amine

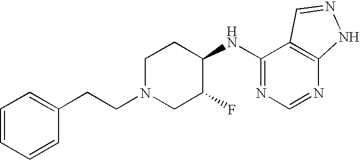

### Step A: benzyl 4-{[tert-butyl(dimethyl)silyl]oxy}-3,6-dihydropyridine-1(2H)-carboxylate

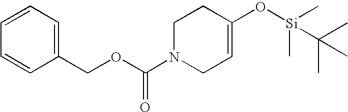

To a solution of benzyl 4-oxopiperidine-1-carboxylate (170 g, 750 mmol) in DMF (400 mL) was added DIPEA (195 mL) and tert-butyl(dimethyl)silyl trifluoromethanesulfonate (220 g, 840 mmol) at room temperature. After 5 hours, the reaction mixture was poured into brine and extracted with ethyl acetate. The combined organics were dried over sodium sulfate, filtered and concentrated to give the title compound (240 g). MS: 348 (M+H⁺).

### Step B: benzyl 3-fluoro-4-oxopiperidine-1-carboxylate

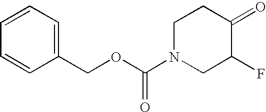

To a solution of benzyl 4-{[tert-butyl(dimethyl)silyl]oxy}-3,6-dihydropyridine-1(2H)-carboxylate (240 g, 700 mmol) in DMF (1.0 L) was added 1-(chloromethyl)-4-fluoro-1,4-diazoniabicyclo[2.2.2]octane ditetrafluoroborate (310 g, 880 mmol) at room temperature. After 2 hours, the reaction mixture was poured into brine and extracted with ethyl acetate. The combined organics were dried over sodium sulfate, filtered and concentrated to give the title compound (251 g). MS: 252 (M+H⁺).

### Step C: cis- and trans-benzyl 3-fluoro-4-hydroxypiperidine-1-carboxylate

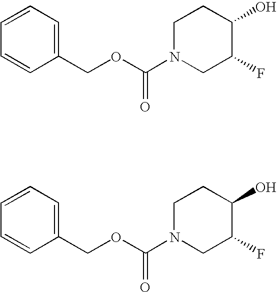

To a solution of benzyl 3-fluoro-4-oxopiperidine-1-carboxylate (24 g, 97 mmol) in methanol (250 mL) was added sodium borohydride (5.5 g, 150 mmol) in small portions at 0° C. at such a rate to prevent the reaction mixture from warming above room temperature. After 4 hours, the reaction mixture was concentrated. The resulting residue was dissolved in ethyl acetate, washed with brine, dried over sodium sulfate, filtered and concentrated to dryness. Purification by silica gel chromatography (10% ethyl acetate/hexane→60% ethyl acetate/hexane) gave both title compounds:

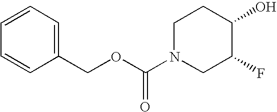

cis-benzyl 3-fluoro-4-hydroxypiperidine-1-carboxylate: 18 g; colorless oil; MS: 254 (M+H⁺); ¹H NMR (400 MHz, CDCl₃): δ 7.40-7.32 (m, 5H), 5.20 (s, 2M), 4.72-4.52 (m, 1H), 4.15-3.25 (m, 6H), 2.17 (s, 1H), 1.88-1.67 (m, 2H);

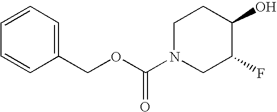

trans-benzyl 3-fluoro-4-hydroxypiperidine-1-carboxylate: 4.5 g; colorless oil; MS: 254 (M+H⁺); ¹H NMR (400 MHz, CDCl₃): δ 7.40-7.32 (m, 5H), 5.18 (s, 21-1), 4.40-3.80 (m, 4H), 3.25-3.05 (m, 2H), 2.30 (s, 1H), 2.00 (s, 1H) 1.58 (s, 1H).

### Step D: cis-benzyl 3-fluoro-4-[(methylsulfonyl)oxy]piperidine-1-carboxylate

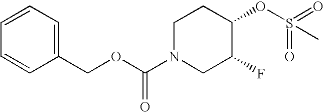

To a solution of cis-benzyl 3-fluoro-4-hydroxypiperidine-1-carboxylate (24 g, 97 mmol) in methylene chloride (150 mL) was added DIPEA (28 mL, 190 mmol) and methanesulfonyl chloride (9.0 g, 79 mmol) at −110° C. and the reaction mixture was allowed to slowly warm to room temperature. After 3 hours, the reaction mixture was poured into brine, extracted with methylene chloride, dried over sodium sulfate, filtered and concentrated to give the title compound (31 g) as an oil. MS: 332 (M+H⁺).

### Step E: Trans-benzyl 4-azido-3-fluoropiperidine-1-carboxylate

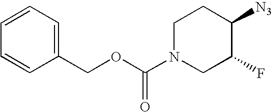

To a solution of cis-benzyl 3-fluoro-4-[(methylsulfonyl)oxy]piperidine-1-carboxylate (10 g, 30 mmol) in DMF (25 mL) was added sodium azide (9.8 g, 150 mmol) and the reaction mixture was heated to 80° C. After 48 hours, the reaction mixture was poured into brine, extracted with ethyl acetate, dried over sodium sulfate, filtered and concentrated. Purification by silica gel chromatography (0% isopropanol/methylene chloride→10% IPA/methylene chloride) gave the title compound (6.6 g) as an oil. MS: 279 (M+H⁺).

### Step F: trans-4-azido-3-fluoropiperidine

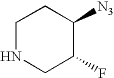

To a solution of trans-benzyl 4-azido-3-fluoropiperidine-1-carboxylate (15 g, 54 mmol) in TFA (5 mL) was added thioanisole (13 g, 110 mmol) and the solution was heated at 70° C. for 1 hour. The mixture was cooled and concentrated. The resulting residue was dissolved in ethyl acetate (60 mL), treated with a saturated solution of HCl in ethyl acetate (50 mL) at room temperature, and concentrated. Recrystallization from ethyl acetate and hexane gave the title compound (6.5 g) as a white solid, MS: 145 (M+H⁺).

### Step G: trans-4-azido-3-fluoro-1-(phenylacetyl)piperidine

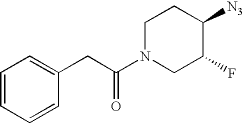

To a solution of trans-4-azido-3-fluoropiperidine (1.2 g, 8.7 mmol) in methylene chloride (25 mL) was added DIPEA (3.8 mL, 26 mmol) and phenylacetyl chloride (1.3 g, 8.7 mmol) at room temperature. After 1 hour, the reaction mixture was poured into saturated sodium bicarbonate and extracted with chloroform. The combined organic layers were dried over sodium sulfate, filtered, and concentrated to give the title compound (1.7 g). MS: 263 (M+H⁺).

### Step H: trans-3-fluoro-1-(2-phenylethyl)piperidin-4-amine

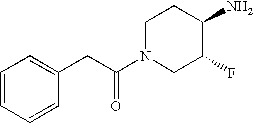

To a solution of trans-4-azido-3-fluoro-1-(phenylacetyl)piperidine (1.7 g, 6.5 mmol) in THF (20 mL) at room temperature under nitrogen was added 1M borane in THF (79 mL, 79 mmol) and the reaction mixture was heated to 80° C. After 1 hour, the reaction mixture was cooled to room temperature, quenched with 6N HCl (50 mL) and heated to 80° C. After 1 hour, the reaction mixture was basified to pH>8 with 5M NaOH and extracted with chloroform. The combined organics were dried over sodium sulfate, filtered, and concentrated to give the title compound (1.0 g). MS: 223 (M+H⁺).

### Step I: N-[trans-3-fluoro-1-(2-phenylethyl)piperidin-4-yl]-1H-pyrazolo[3,4-d]pyrimidin-4-amine

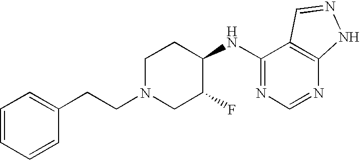

The title compound was prepared using procedures similar to that reported in Example 1, Step C above. HRMS (M+H⁺): calculated=341.1878, observed=341.1885; ¹H NMR (400 MHz, CD₃OD): δ 8.30 (s, 1H), 8.15 (s, 1H), 7.38-7.18 (m, 5H), 4.70-4.40 (m, 2H), 3.40 (m, 1H), 3.00 (m, 1H), 2.85-2.58 (m, 4H), 2.30 (m, 2H), 2.25 (m, 1H), 1.60 (m, 1H).

### EXAMPLE 29

### N-[cis-3-fluoro-1-(2-phenylethyl)piperidin-4-yl]-1H-pyrazolo[3,4-d]pyrimidin-4-amine

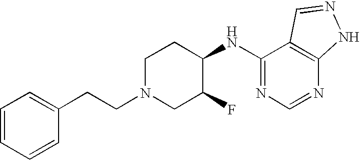

### Step A: cis-4-azido-3-fluoropiperidine

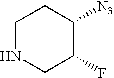

The title compound was prepared from trans-benzyl 3-fluoro-4-hydroxypiperidine-1-carboxylate (Example 28, Step C) using procedures similar to that reported in Example 28, Steps D, E and F above. MS: 145 (M+H⁺).

### Step B: N-[cis-3-fluoro-1-(2-phenylethyl)piperidin-4-yl]-1H-pyrazolo[3,4-d]pyrimidin-4-amine

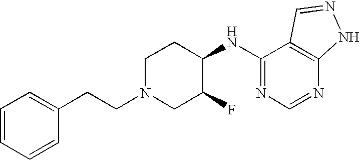

The title compound was prepared using procedures similar to that reported in Example 28, Steps G, H and I above. HRMS (M+H⁺): calculated=341.1876, observed=341.1885; ¹H NMR (400 MHz, CD₃OD) 8.30-8.20 (m, 2H), 7.32-7.18 (m, 5H), 5.00-4.80 (m, 1H), 4.50-4.40 (m, 1H), 3.40 (m, 1H), 3.10 (m, 1H), 2.85-2.60 (m, 4H), 2.50-2.10 (m, 3H), 1.90 (m, 1H).

### EXAMPLE 30

### N-{trans-3-fluoro-1-[2-(4-methylphenyl)ethyl]piperidin-4-yl}-1H-pyrazolo[3,4-d]pyrimidin-4-amine

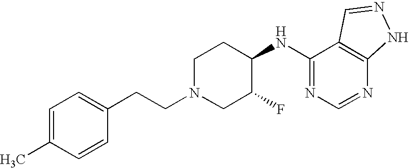

The title compound was prepared using procedures similar to that reported in Example 28, Steps G, H and I above. HRMS (M+HI): calculated=355.4317, observed=355.4216; ¹HNMR (400 MHz, CD₃OD) 8.65 (s, 2H), 7.30-7.18 (m, 4H), 4.58-4.20 (m, 2H), 4.15-3.85 (m, 1H), 3.80-3.35 (m, 7H), 3.15 (m, 2H), 2.38 (s, 3H).

### EXAMPLE 31

### N-{cis-3-fluoro-1-[2-(4-methylphenyl)ethyl]piperidin-4-yl}-1H-pyrazolo[3,4-d]pyrimidin-4-amine

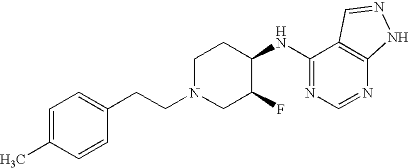

The title compound was prepared using procedures similar to that reported in Example 29 above. HRMS (M+H⁺): calculated=355.4317, observed=355.4128; ¹HNMR (400 MHz, CD₃OD) 8.62 (s, 2H), 7.28-7.20 (m, 4H), 5.38-4.80 (m, 1H), 4.20-3.83 (m, 1H), 3.85-3.42 (m, 71), 3.25 (m, 2H), 2.35 (s, 3H).

### EXAMPLE 32

### N-[trans-3-fluoro-1-(2-fluoro-2-phenylethyl)piperidin-4-yl]-1H-pyrazolo[3,4-d]pyrimidin-4-amine

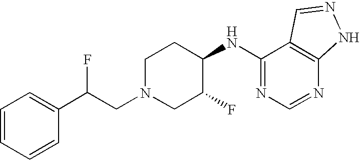

### Step A: 2-[trans-4-azido-3-fluoropiperidin-1-yl]-1-phenylethanol

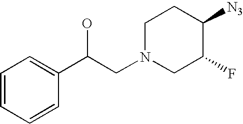

The title compound was prepared using procedures similar to that reported in Example 6, Step A above. MS: 265 (M+H⁺).

### Step B: trans-4-azido-3-fluoro-1-(2-fluoro-2-phenylethyl)piperidine

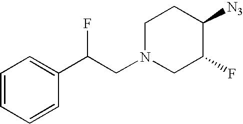

The title compound was prepared using procedures similar to that reported in Example 17, Step A above. MS: 267 (M+H⁺).

### Step C: trans-3-fluoro-1-(2-fluoro-2-phenylethyl)piperidin-4-amine

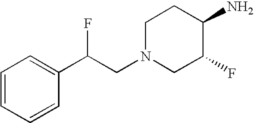

The title compound was prepared using procedures similar to that reported in Example 28, Step H above. MS: 241 (M+H⁺).

### Step D: N-[trans-3-fluoro-1-(2-fluoro-2-phenylethyl)piperidin-4-yl]-1H-pyrazolo[3,4-d]pyrimidin-4-amine

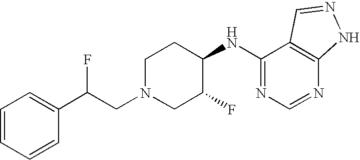

The title compound was prepared using procedures similar to that reported in Example 1, Step C above. HRMS (M+H⁺): calculated=359.1780, observed=359.1791; ¹H NMR (400 MHz, CD₃OD): δ 8.25-8.15 (m, 2H), 7.40-7.35 (m, 5H), 5.80-5.60 (m, 1H), 4.80-4.35 (m, 2H), 3.45 (m, 1H), 3.10-2.95 (m, 2H), 2.85-2.70 (m, 1H), 2.40 (m, 2H), 2.18 (m, 1H), 1.70 (m, 1H).

### EXAMPLE 33

### N-[trans-1-(2,2-difluoro-2-phenylethyl)-3-fluoropiperidin-4-yl]-1H-pyrazolo[3,4-d]pyrimidin-4-amine

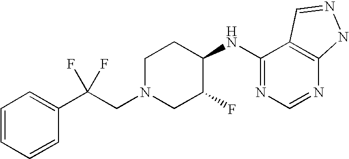

### Step A: trans-4-azido-1-[difluoro(phenyl)acetyl]-3-fluoropiperidine

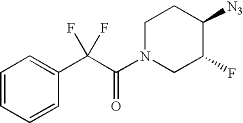

To a solution of difluoro(phenyl)acetic acid (Hagele, G., Haas, A. J. of Fluorine Chemistry (1996) 76, 15-19) (1.7 g, 10.4 mmol) in methylene chloride (25 mL) was added oxalylchloride (5.2 mL, 2M in methylene chloride, 10.4 mmol) and a drop of DMF at room temperature. After 1 hour, the reaction mixture was added to a suspension of trans-4-azido-3-fluoropiperidine (1.5 g, 10 mmol) and resin-DIPEA (40 mmol eq) in methylene chloride (50 mL) at room temperature. After 2 hour, the reaction mixture was filtered and concentrated to yield the title compound (2.5 g) as a solid. MS: 299 (M+H⁺).

### Step B: N-[(3R,4R)-1-(2,2-difluoro-2-phenylethyl)-3-fluoropiperidin-4-yl]-1H-pyrazolo[3,4-d]pyrimidin-4-amine

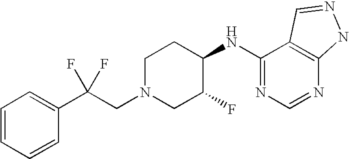

The title compound was prepared from trans-4-azido-1-[difluoro(phenyl)acetyl]-3-fluoropiperidine according to the procedure reported in Example 28, Steps H and I presented above. HRMS (M+H⁺): calculated=377.1676, observed=377.1696; ¹HNMR (400 MHz, CD₃OD): δ 8.20 (s, 1H), 8.10 (s, 1H), 7.55-7.45 (m, 5H), 4.51-4.31 (m, 2H), 3.17-3.10 (m, 3H), 2.80 (m, 2H), 2.52 (m, 2H), 2.00 (m, 1H), 1.51 (m, 1H).

### EXAMPLE 34

### N-[cis-1-(2,2-difluoro-2-phenylethyl)-3-fluoropiperidin-4-yl]-1H-pyrazolo[3,4-d]pyrimidin-4-amine

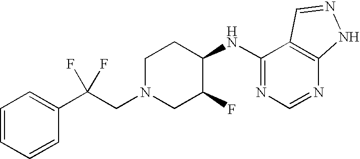

The title compound was prepared from cis-4-azido-3-fluoropiperidine (Example 29, Step A) using procedures similar to that reported iii Example 33 above. HRMS (M+H⁺): calculated=377.1676, observed=377.1696; ¹HNMR (400 MHz, CD₃OD): δ 8.22 (m, 2H), 7.56 (m, 2H), 7.44 (m, 3H), 4.84-4.70 (m, 1H), 4.39-4.33 (m, 1H), 3.31-3.12 (m, 3H), 2.87-2.57 (m, 3H), 2.07 (m, 1H), 1.70 (m, 1H).

### EXAMPLE 35

### N-{trans-1-[2,2-difluoro-2-(4-methylphenyl)ethyl]-3-fluoropiperidin-4-yl}-1H-pyrazolo[3,4-d]pyrimidin-4-amine

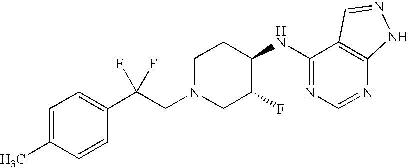

The title compound was prepared using procedures similar to that reported in Example 33 above. HRMS (M+H⁺): calculated=391.1853, observed=391.1811; ¹HNMR (400 MHz, CD₃OD): δ 8.22 (s, 1H), 8.12 (s, 1H), 7.55-7.22 (m, 4H), 4.58-4.30 (m, 2H), 3.30-3.12 (m, 3H), 2.78 (m, 1H), 2.52 (m, 2H), 2.42 (s, 3H), 1.98 (m, 1H), 1.62 (m, 1H).

### EXAMPLE 36

### N-(trans-1-{2,2-difluoro-2-[4-(trifluoromethyl)phenyl]ethyl}-3-fluoropiperidin-4-yl)-1H-pyrazolo[3,4-d]pyrimidin-4-amine

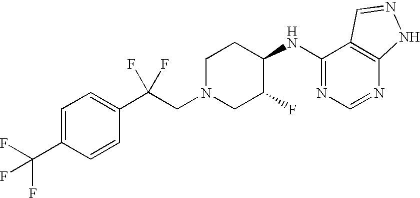

The title compound was prepared using procedures similar to that reported in Example 33 above. HRMS (M+H⁺): calculated=445.1570, observed=445.1567; ¹HNMR (400 MHz, CD₃OD): δ 8.31-8.22 (m, 2H), 7.81-7.62 (m, 4H), 4.42-4.20 (m, 1H), 3.21 (m, 2H), 2.89-2.62 (m, 4H), 2.04 (m, 2H), 1.73 (m, 1H).

### EXAMPLE 37

### N-(cis-1-{2,2-difluoro-2-[4-(trifluoromethyl)phenyl]ethyl}-3-fluoropiperidin-4-yl)-1H-pyrazolo[3,4-d]pyrimidin-4-amine

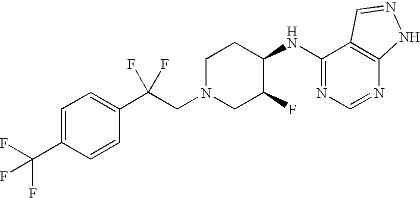

The title compound was prepared using procedures similar to that reported in Example 34 above. HRMS (M+H⁺): calculated=445.1570, observed=445.1539; ¹HNMR (400 M, CD₃OD): δ 8.70 (s, 1H), 8.62 (s, 1H), 7.82 (m, 4H), 5.18-5.12 (m, 1H), 3.88-3.78 (m, 4H), 3.52-3.19 (m, 2H), 2.42 (m, 2H), 2.08 (m, 1H).

### EXAMPLE 38

### N-[1-(2,2-difluoro-2-phenylethyl)azepan-4-yl]-1H-pyrazolo[3,4-d]pyrimidin-4-amine

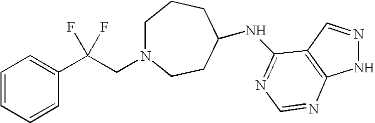

### Step A: 1-[difluoro(phenyl)acetyl]azepan-4-one

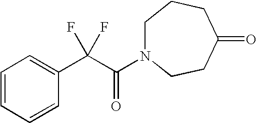

The title compound was prepared using procedures similar to that reported ii Example 19, Step B above. MS: 268 (M+H⁺).

### Step B: N-[1-(2,2-difluoro-2-phenylethyl)azepan-4-yl]-1H-pyrazolo[3,4-d]pyrimidin-4-amine

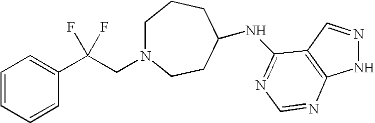

The title compound was prepared using procedures similar to that reported in Example 28, Step C, D, E, H and I above. HRMS (M+H⁺): calculated=373.1947, observed=373.1955; ¹H NMR (400 MHz, CD₃OD): δ 8.67 (s, 1H), 8.57 (s, 1H), 7.66-7.55 (m, 5H), 4.72 (m, 1H), 4.19-4.10 (m, 2H), 3.89-3.65 (m, 4H), 2.42-2.34 (m, 3H), 2.21-2.08 (m, 1H), 1.98-1.90 (m, 1H). Purification by chiral HPLC (15% isopropanol/hexanes/0.1% DEA→20% isopropanol/hexanes/0.1% DEA; Chiralpak AD, commercially available from Chiral Technologies, Inc., Exton, Pa.) provided optically pure products, which were treated with anhydrous hydrochloric acid in ethyl acetate solution and concentrated to give the hydrochloride salts of the title compounds: (R or S)-N-[1-(2,2-difluoro-2-phenylethyl)azepan-4-yl]-1H-pyrazolo[3,4-d]pyrimidin-4-amine: Peak 1, HRMS (M+H⁺): calculated=373.1947, observed=373.1953; (S or R)-N-{1-[2,2-difluoro-2-(4-methylphenyl)ethyl]azepan-4-yl}-1H-pyrazolo[3,4-d]pyrimidin-4-amine: Peak 2, HRMS (M+H⁺): calculated=373.1947, observed=373.1958.

### EXAMPLE 39

### N-[1-(2,2-difluoro-2-(4-methylphenyl)ethyl)azepan-4-yl]-1H-pyrazolo[3,4-d]pyrimidin-4-amine

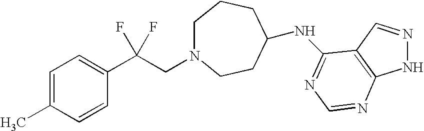

The title compound was prepared using procedures similar to that reported in Example 38 above. Purification by chiral HPLC (Chiralcel OJ, 60% isopropanol/hexanes/0.1% DEA) gave the enantiomers of the title compound: (R or S)-N-{1-[2,2-difluoro-2-(4-methylphenyl)ethyl]azepan-4-yl}-1H-pyrazolo[3,4-c]pyrimidin-4-amine: Peak 1, HRMS (M+H⁺): calculated=387.2104, observed=387.2075; ¹H NMR (400 MHz, CD₃OD): δ 8.22 (s, 1H), 8.10 (s, 1H), 7.43 (d, J=6.4 Hz, 2H), 7.27 (d, J=6.4 Hz, 2H), 4.38 (m, 1H), 3.20 (t, J=11.4 Hz, 2H), 2.88-2.77 (m, 4H), 2.38 (s, 3H), 1.99-1.96 (m, 1H), 1.80-1.71 (m, 3H), 1.64-1.62 (m, 1H); (S or R)-N-{1-[2,2-difluoro-2-(4-methylphenyl)ethyl]azepan-4-yl}-1H-pyrazolo[3,4-d]pyrimidin-4-amine: Peak 2, HRMS (M+H⁺): calculated=387.2104, observed=387.2104; ¹H NMR (400 MHz, CD₃OD): δ 8.22 (s, 1H), 8.10 (s, 1H), 7.43 (d, J=6.4 Hz, 2H), 7.27 (d, J=6.4 Hz, 2H), 4.38 (m, 1H), 3.20 (t, J=11.4 Hz, 2H), 2.88-2.77 (m, 4H), 2.38 (s, 3H), 1.99-1.96 (m, 1H), 1.80-1.71 (m, 3H), 1.64-1.62 (m, 1H).

### EXAMPLE 40

### N-{exo-3-[2,2-difluoro-2-(4-methylphenyl)ethyl]-3-azabicyclo[3.1.0]hex-6-yl}-1H-pyrazolo[3,4-d]pyrimidin-4-amine

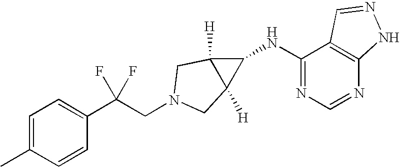

The title compound was prepared from tert-butyl exo-3-azabicyclo[3.1.0]hex-6-ylcarbamate (prepared by literature procedure: Norris, T., Braish, T. F., Butters, M., DeVries, K. M., Hawkins, J. M., Massett, S. S., Rose, P. R., Santafianos, D., Sklavounos, C. J. Chem. Soc., Perkin Trans. 1 (2000) 1615-1622) using procedures similar to that reported in Example 19 above. HRMS (M+H⁺): calculated=371.1791, observed=371.1801; ¹H NMR (400 MHz, CD₃OD): δ 8.62-8.57 (s, 1H), 8.54-8.49 (s, 1H), 7.51-7.44 (m, 2H), 7.39-7.32 (m, 2H), 4.16-3.80 (m, 3), 3.63-3.56 (m, 1H), 2.51-2.42 (m, 2H), 2.42-2.36 (s, 3H).

### EXAMPLE 41

### Racemic-N-[(3aS*,4R*,6aR*)-2-(2-phenylethyl)octahydrocyclopenta[c]pyrrol-4-yl]-1H-pyrazolo[3,4-d]pyrimidin-4-amine

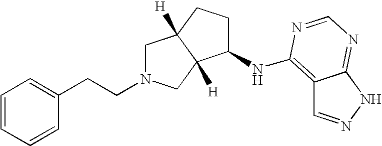

### Step A: racemic-2-benzylhexahydrocyclopenta[c]pyrrol-4(1H)-one

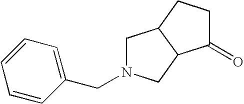

To a solution of N-benzyl-1-methoxy-N-[(trimethylsilyl)methyl]methenamine (11 g, 46 mmol) and cyclopent-2-en-1-one (3.8 g, 46 mmol) in methylene chloride (50 mL) was added TFA (12 mL, 68 mmol) at room temperature. After 1 hour, the reaction mixture was poured into 1M NaOH (500 mL) and extracted with ethyl acetate. The combined organics were dried over sodium sulfate, filtered and concentrated to dryness. The resulting residue was dissolved in ethyl acetate, treated with a saturated solution of HCl in ethyl acetate and concentrated to give the HCl salt of the title compound (11 g) as a solid. MS: 216 (M+H⁺).

### Step B: racemic-(3aS*,4S*,6aR*)-2-benzyloctahydrocyclopenta[c]pyrrol-4-ol

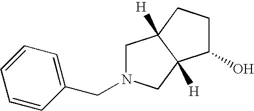

To a solution of racemic-2-benzylhexahydrocyclopenta[c]pyrrol-4(1H)-one (10 g, 46 mmol) in THF (50 mL) was added L-Selectride (8.8 g, 46 mmol, commercially available from Sigma-Aldrich, Inc., St. Louis, Mo.) at −78° C. and the reaction mixture was allowed to slowly warm to room temperature. After 2 hours, the reaction mixture was poured into 1M NaOH (500 mL) and extracted with ethyl acetate. The combined organics were dried over sodium sulfate, filtered and concentrated. Purification by silica gel chromatography (0% IPA/methylene chloride→10% isopropanol/methylene chloride) gave the title compound (5.0 g). MS: 218 (M+H⁺).

### Step C: racemic-tert-butyl (3aS*,4S*,6aR*)-4-hydroxyhexahydrocyclopenta[c]pyrrole-2(1H)-carboxylate

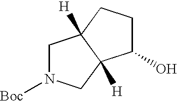

To a solution of racemic-(3aS*,4S*,6aR*)-2-benzyloctahydrocyclopenta[c]pyrrol-4-ol (1.4 g, 6.2 mmol) in ethanol (100 mL) was added di-tert-butyl dicarbonate (1.5 g, 7.0 mmol) and palladium hydroxide, 20 wt % Pd (dry basis) on carbon, wet (commercially available from Sigma-Aldrich, Inc., St. Louis, Mo.) and the reaction mixture was placed under 50 psi of hydrogen at room temperature. After 20 hours, the reaction mixture was filtered and concentrated to give the title compound (0.85 g). MS: 228 (M+H⁺).

### Step D: racemic-tert-butyl (3aS*,4R*,6aR*)-4-azidohexahydrocyclopenta[c]pyrrole-2(1H)-carboxylate

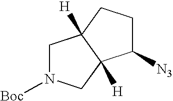

The title compound was prepared using procedures similar to that reported in Example 28, Steps D and E above. MS: 253 (M+H⁺).

### Step E: Racemic-N-[(3aS,4R,6aR)-2-(2-phenylethyl)octahydrocyclopenta[c]pyrrol-4-yl]-1H-pyrazolo[3,4-d]pyrimidin-4-amine

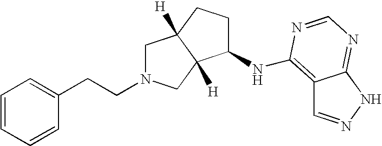

The title compound was prepared using procedures similar to that reported in Example 28, Steps G, H and I above. HRMS (M+H⁺): calculated=349.2135, observed=349.2146; ¹HNMR (400 MHz, CD₃OD): δ 8.32 (s, 1H), 8.20 (s, 1H), 7.35-7.15 (m, 5H), 4.38 (s, 1H), 2.89-2.60 (m, 10H), 2.38 (s, 1H), 2.20-2.08 (m, 2H), 1.70 (m, 1H), 1.50 (m, 1H).

### EXAMPLE 42

### Racemic-N-[(3aS*,4R*,6aR*)-2-(2,2-difluoro-2-phenylethyl)octahydrocyclopenta[c]pyrrol-4-yl]-1H-pyrazolo[3,4-d]pyrimidin-4-amine

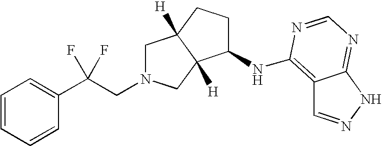

The title compound was prepared from racemic-(3aS*,4R*,6aR*)-4-azidooctahydrocyclopenta[c]pyrrole (see Example 41, Step D) using procedures similar to that reported in Example 33 above. HRMS (M+H⁺): calculated=385.1947, observed=385.1923; ¹HNMR (400 MHz, CD₃OD): δ 8.28 (s, 1H), 8.15 (s, 1H), 7.60 (s, 2H), 7.38 (m, 3H), 4.32 (s, 1H), 3.10 (m, 2H), 2.80-2.42 (m, 6H), 2.00 (m, 2H), 1.52 (m, 1H), 1.40 (m, 1H).

### EXAMPLE 43

### Racemic-N-{(3aS*,4R*,6aR*)-2-[2,2-difluoro-2-(4-methylphenyl)ethyl]octahydrocyclopenta[c]pyrrol-4-yl}-1H-pyrazolo[3,4-d]pyrimidin-4-amine

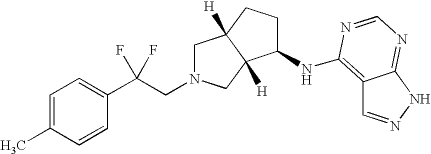

The title compound was prepared from racemic-(3aS*,4R*,6aR*)-4-azidooctahydrocyclopenta[c]pyrrole (see Example 41, Step E) using procedures similar to that reported in Example 33 above. HRMS (M+H⁺): calculated=399.2104, observed=399.2095; ¹HNMR (400 MHz, CD₃OD): δ 8.82 (s, 1H), 8.60 (s, 1H), 7.58-7.40 (m, 4H), 4.70 (s, 1H), 4.28-4.10 (m, 4H), 3.29-3.08 (m, 3H), 2.50-2.30 (m, 4H), 2.28-2.00 (m, 2H), 1.80 (m, 1H).

### EXAMPLE 44

### Endo-N-[8-(2,2-difluoro-2-phenylethyl)-8-azabicyclo[3.2.1]oct-3-yl]-1H-pyrazolo[3,4-d]pyrimidin-4-amine

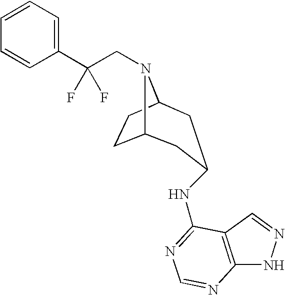

### Step A: tert-butyl 3-(hydroxyimino)-8-azabicyclo[3.2.1]octane-8-carboxylate

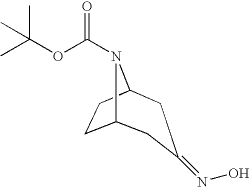

To a solution of tert-butyl 3-oxo-8-azabicyclo[3.2.1]octane-8-carboxylatedifluoro (3.0 g, 13 mmol) in methanol (35 mL) was added hydroxylamine hydrochloride (4.6 g, 67 mmol) and sodium acetate (11 g, 133 mmol) at room temperature. After 72 hours, the mixture was poured into water and extracted with ether. The combined organics were washed with brine, dried over sodium sulfate, filtered and concentrated to give the title compound (3.1 g) as a white solid. MS: 241 (M+H⁺).

### Step B: endo-tert-butyl 3-amino-8-azabicyclo[3.2.1]octane-8-carboxylate

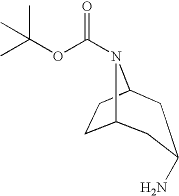

The following procedure is based on a method reported by Suzuki, M., Ohuchi, Y., Asanuma, H., Kaneko, T., Yokomori, S., Ito, C., Isobe, Y., Muramatsu, M. Chem. Pharm. Bull. (2001) 49(1), 29-39. To a solution of tert-butyl 3-(hydroxyimino)-8-azabicyclo[3.2.1]octane-8-carboxylate (2.0 g, 8.3 mmol) in acetic acid (25 mL) was added platinum dioxide (0.10 g) and the solution was exposed to 50 psi hydrogen gas on a Parr hydrogenation apparatus (commercially available from Parr Instrument Company, Moline, Ill.) After 17 hours, the mixture was filtered and concentrated. The residue was poured into 5M NaOH and extracted with methylene chloride. The combined organics were washed with brine, dried over sodium sulfate, filtered and concentrated to give the title compound (2.0 g) as an oil.

MS: 227 (M+H⁺).

### Step C: Endo-tert-butyl 3-{[(benzyloxy)carbonyl]amino}-8-azabicyclo[3.2.1]octane-8-carboxylate

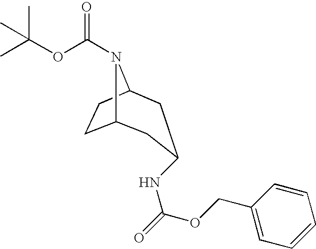

To a solution of exo-tert-butyl 3-amino-8-azabicyclo[3.2.1]octane-8-carboxylate (2.0 g, 8.7 mmol) in methylene chloride (18 mL) was added DIPEA (3.0 mL, 17 mmol) and benzylchloroformate (1.1 mL, 13 mmol) at room temperature. After 2 hours, the reaction mixture was diluted with ethyl acetate, washed with water, 1M citric acid, water, 1M NaOH, brine, dried over sodium sulfate, filtered and concentrated to give the title compound (3.3 g). MS: 361 (M+H⁺).

### Step D: Endo-benzyl 8-azabicyclo[3.2.1]oct-3-ylcarbamate

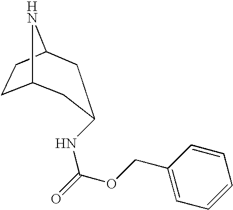

The title compound was prepared using procedures similar to that reported in Example 1, Step B above. MS: 261 (M+H⁺).

### Step E: Endo-benzyl {8-[difluoro(phenyl)acetyl]-8-azabicyclo[3.2.1]oct-3-yl}carbamate

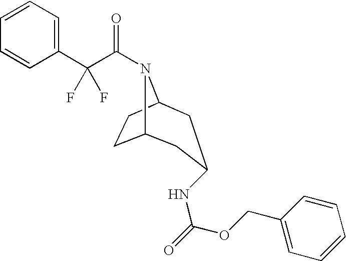

The title compound was prepared using procedures similar to that reported in Example 19, Step B above. MS: 415 (M+H⁺).

### Step F: Endo-N-[8-(2,2-difluoro-2-phenylethyl)-8-azabicyclo[3.2.1]oct-3-yl]-1H-pyrazolo[3,4-d]pyrimidin-4-amine

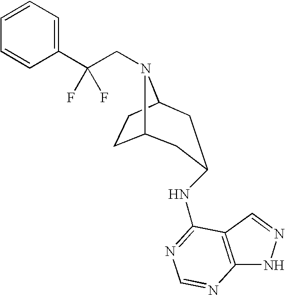

The title compound was prepared using procedures similar to that reported in Example 28, Steps F, H and I above. HRMS (M+H⁺): calculated=385.1947, observed=385.1933; ¹H NMR (400 MHz, CD₃OD): δ 8.88 (s, 1H), 8.66 (s, 1H), 7.69-7.67 (m, 2H), 7.61-7.57 (m, 3H), 4.60 (m, 1H), 4.31 (br s, 2H), 4.00 (m, 2H), 2.75 (m, 2H), 2.59-2.45 (m, 6H).

### EXAMPLE 45

### Exo-N-[8-(2,2-difluoro-2-phenylethyl)-8-azabicyclo[3.2.1]oct-3-yl]-1H-pyrazolo[3,4-d]pyrimidin-4-amine

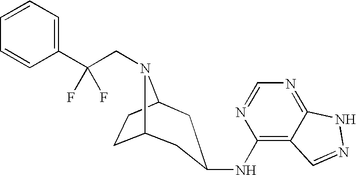

### Step A: exo-tert-butyl 3-amino-8-azabicyclo[3.2.1]octane-8-carboxylate

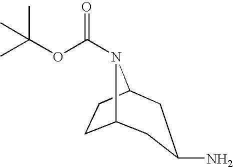

To a solution of tert-butyl 3-(hydroxyimino)-8-azabicyclo[3.2.1]octane-8-carboxylate (1.0 g, 4.2 mmol; Example 44, Step A) in 1-propanol (15 mL, dried over 4 Å molecular sieves) was added sodium metal (1.0 g, 42 mmol) in small portions over a 10 minute period at room temperature under an atmosphere of nitrogen. The reaction mixture was heated to reflux for 4.5 hours and cooled to room temperature. The reaction mixture was quenched with water and extracted with methylene chloride. The combined organics were extracted with 2M HCl, and the combined aqueous layers were basified with solid potassium hydroxide and extracted with methylene chloride. The combined organics were dried over sodium sulfate, filtered and concentrated to give the title compound (0.36 g) as a colorless oil. MS: 227 (M+H⁺).

### Step B: Exo-N-[8-(2,2-difluoro-2-phenylethyl)-8-azabicyclo[3.2.1]oct-3-yl]-1H-pyrazolo[3,4-d]pyrimidin-4-amine

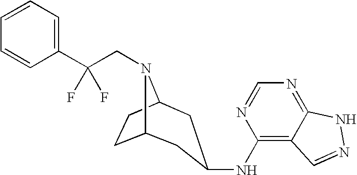

The title compound was prepared using procedures similar to that reported in Example 44, Step C, D, E and F above. HRMS (M+H⁺): calculated=385.1947, observed=385.1960; ¹H NMR (400 MHz, CD₃OD): δ 8.18 (s, 1H), 8.07 (s, 1H), 7.57-7.55 (m, 2H), 7.45-7.43 (m, 3H), 4.49-4.48 (m, 1H), 3.14 (br s, 2H), 3.06 (t, J=13.6 Hz, 2H), 1.95-1.93 (m, 2H), 1.80-1.76 (m, 4H), 1.70-1.64 (m, 2H).

### EXAMPLE 46

### N-(1-{2-[4-(Difluoromethyl)phenyl]-2,2-difluoroethyl}piperidin-4-yl)-1H-pyrazolo[3,4-d]pyrimidin-4-amine

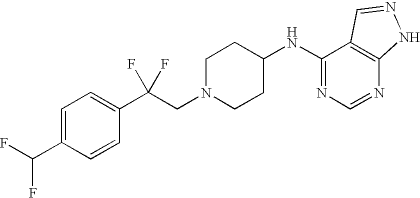

The title compound was prepared using procedures similar to that reported in Example 19 above. HRMS (M+H⁺): calculated=409.1759, observed=4019.1725; ¹H NMR (500 MHz, CD₃OD): δ 8.74-8.65 (s, 1H), 8.65-8.58 (s, 1H), 7.83-7.73 (m, 4H), 7.01-6.75 (m, 1H), 4.77-4.64 (m, 1H), 4.22-4.07 (m, 2H), 3.98-3.87 (m, 2H), 3.58-3.44 (m, 2H), 2.48-2.37 (m, 2H), 2.29-2.15 (m, 2H).

### EXAMPLE 47

### N-{1-[2-(4-Ethylphenyl)-2,2-difluoroethyl]piperidin-4-yl}-1H-pyrazolo[3,4-d]pyrimidin-4-amine

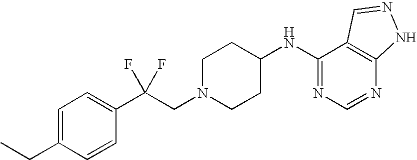

To a solution of N-{1-[2-(4-bromophenyl)-2,2-difluoroethyl]piperidin-4-yl}-1-(tetrahydro-2H-pyran-2-yl)-1H-pyrazolo[3,4-d]pyrimidin-4-amine (Example 46, 0.21 g, 0.39 mmol) in anhydrous toluene (1.5 mL) was added ethylboronic acid (0.04 g, 0.59 mmol), anhydrous potassium phosphate; tribasic (0.25 g, 1.18 mmol), Bis(dibenzylideneacetone) palladium (Pd(dba)₂) (0.01 g, 0.02 mmol), and 1,2,3,4,5-Pentaphenyl-1′-(di-tert-butylphosphino)ferrocene (Q-phos) (0.028 g, 0.04 mmol). the solution was heated at 120° C. for 48 hours while stirring capped in a hot oil bath. The mixture was cooled, dissolved in ethyl acetate (30 mL) washed with 1M NaOH, then water, and, finally, brine. The combined organics were dried over sodium sulfate, filtered and concentrated to dryness. The resulting residue was dissolved in methanol (2.5 mL) and purified by reverse phase chromatography (5% acetonitrile/0.1% trifluoroacetic acid/water→95% acetonitrile/0.1% trifluoroacetic acid/water, XTerra® MSC8 column, commercially available from Waters Corporation, Milford, Mass.). The desired fractions were concentrated, and the resulting residue was dissolved in methanol (3 mL) and treated with a saturated solution of HCl in ethyl acetate (10 mL) at room temperature. After 1 hour solid had precipitated out of solution, which was then filtered to yield the hydrochloride salt of the title compound (0.014 g) as a yellow solid. HRMS (M+H⁺): calculated=387.2104, observed=387.2055; ¹H NMR (500 MHz, CD₃OD): δ 8.64-8.54 (m, 2H), 7.57-7.51 (m, 2H), 7.43-7.38 (m, 2H), 4.72-4.60 (m, 1H), 4.14-3.98 (m, 2H), 3.96-3.82 (m, 2H), 3.54-3.39 (m, 2H), 2.77-2.69 (m, 2H), 2.46-2.36 (m, 2H), 2.26-2.12 (m, 2H), 1.30-1.22 (m, 3H).

### EXAMPLE 48

### N-((3S,4S)-1-{2-[4-(difluoromethyl)phenyl]-2,2-difluoroethyl}-3-fluoropiperidin-4-yl)-1H-pyrazolo[3,4-d]pyrimidin-4-amine

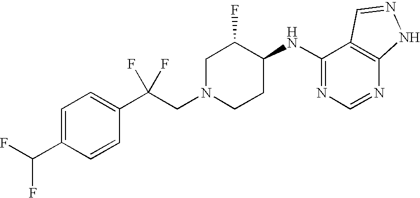

The title compound was prepared using procedures similar to that reported in Example 33 above. MS (M+H⁺)=426.2; ¹H NMR (500 MHz, CD₃OD): δ 8.77 (s, 1H), 8.62 (s, 1H), 7.74 (q, J=8.3 Hz, 4H), 6.86 (t, J=55.9 Hz, 1H), 3.86 (m, 3H), 3.59 (br s, 1H), 3.22 (br, s, 2H), 2.40 (br s, 1H), 2.03 (s, 1H).

### EXAMPLE 49

### N-((3S,4S)-3-fluoro-1-{2-[4-(trifluoromethyl)phenyl]ethyl}piperidin-4-yl)-1H-pyrazolo[3,4-d]pyrimidin-4-amine

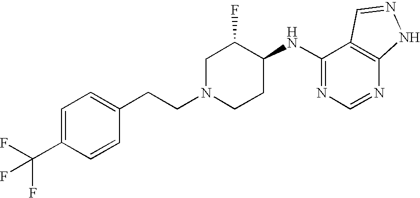

The title compound was prepared using procedures similar to that reported in Example 33 above. MS (M+H⁺) 409.2; ¹H NMR (500 MHz, CD₃OD): δ 8.72 (s, 1H), 8.66 (s, 1H), 7.68 (d, J=8.1 Hz, 2H), 7.56 (d, J=7.8 Hz, 2H), 5.25 (m, 1H), 5.05 (s, 1H), 4.11 (s, 1H), 3.97 (s, 1H), 3.56 (s, 2H), 3.39 (s, 1H), 2.54 (s, 1H), 2.38-2.18 (m, 1H), 2.04 (s, 1H).

### EXAMPLE 50

### N-(1-{2,2-difluoro-2-[4-(trifluoromethyl)phenyl]ethyl}azepan-4-yl)-1H-pyrazolo[3,4-d]pyrimidin-4-amine

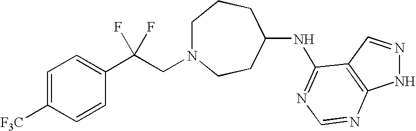

The title compound was prepared using procedures similar to that reported in Example 38 above. Purification by chiral HPLC (20% isopropanol/80% hexanes/0.1% DEA, Chiralpak AD, commercially available from Chiral Technologies, Inc., Exton, Pa.) gave the enantiomers of the title compound: (R or S)-N-(1-{2,2-difluoro-2-[4-(trifluoromethyl)phenyl]ethyl}azepan-4-yl)-1H-pyrazolo[3,4-d]pyrimidin-4-amine: Peak 1, HRMS (M+H⁺): calculated=441.1821, observed=441.1782; ¹H NMR (400 MHz, CD₃OD): δ 8.21 (s, 1H), 8.09 (s, 1H), 7.78 (s, 4H), 4.33 (m, 1H), 3.27 (m, 2H), 2.85-2.76 (m, 4H), 1.97-1.94 (m, 2H), 1.72-1.59 (m, 4H); (S or R)-N-(1-{2,2-difluoro-2-[4-(trifluoromethyl)phenyl]ethyl}azepan-4-yl)-1H-pyrazolo[3,4-d]pyrimidin-4-amine: Peak 2, HRMS (M+H⁺): calculated=441.1821, observed=441.1821; ¹H NMR (400 MHz, CD₃OD): δ 8.21 (s, 1H), 8.09 (s, 1H), 7.78 (s, 4H), 4.33 (m, 1H), 3.27 (m, 2H), 2.85-2.76 (m, 4H), 1.97-1.94 (m, 2H), 1.72-1.59 (m, 4H).

1. A compound of the formula (I): 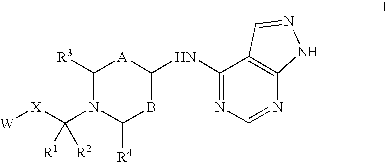
and/or pharmaceutically acceptable salts, individual enantiomers and stereoisomers thereof, wherein:
W is aryl or heteroaryl, wherein said aryl or heteroaryl is optionally substituted with 1-5 substituents independently selected from the group consisting of halogen, C₃₋₆ cycloalkyl, cyano, C₁₋₄ alkoxy, and C₁₋₆ alkyl, wherein said alkoxy is optionally substituted with one or more halogen, and said alkyl is optionally substituted with one or more substituents selected from the group consisting of hydrogen, halogen and hydroxyl;
X is absent or is selected from the group consisting of C₁₋₄alkoxy and C₁₋₃alkyl optionally substituted with one or more substituents selected from the group consisting of hydrogen, halogen, hydroxyl, (═O), and cyano;
A is a bond or C₁₋₃ alkyl, optionally substituted with one or more substituents selected from the group consisting of hydrogen, halogen, hydroxyl, and C₁₋₃ alkyl, wherein said alkyl is optionally substituted with one or more substituents selected from the group consisting of halogen, hydroxyl, C₁₋₄ alkoxy, and cyano;
B is C₁ alkyl, optionally substituted with one or more substituents selected from the group consisting of hydrogen, halogen, hydroxyl, and C₁₋₃ alkyl, wherein said alkyl is optionally substituted with one or more substituents selected from the group consisting of halogen, hydroxyl, C₁₋₄ alkoxy, and cyano, where a ring is formed comprising A and B, where an individual carbon atom in A and an individual carbon atom in B optionally can join to bridge said ring;
R¹ and R² each is independently selected from the group consisting of hydrogen and C₁₋₃ alkyl; and
R³ and R⁴ each is independently selected from the group consisting of hydrogen, hydroxyl, cyano, and C₁₋₃ alkyl, wherein said alkyl is optionally substituted with one or more substituents selected from the group consisting of halogen, hydroxyl, C₁₋₄ alkoxy, and cyano,
where R³ and R⁴, along with the ring to which they are attached, optionally can join to form a bridged cycloalkyl.

2. The compound of claim 1, and/or an individual enantiomer, diastereomer or a pharmaceutically acceptable salt thereof, wherein:
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;
X is selected from the group consisting of C₁₋₄alkoxy and C₁₋₃ alkyl optionally substituted with one or more substituents selected from the group consisting of hydrogen, halogen, hydroxyl and (═O);
R¹ and R² each is independently selected from the group consisting of hydrogen and C₁₋₃ alkyl;
R³ and R⁴ each is independently hydrogen;
A is a bond or C₁₋₃ alkyl, optionally substituted with one or more substituents selected from the group consisting of hydrogen and halogen; and
B is C₁alkyl, optionally substituted with one or more substituents selected from the group consisting of hydrogen and halogen, where a ring is formed comprising A and B, where an individual carbon atom in A and an individual carbon atom in B optionally can join to bridge said ring.

3. A compound of claim 1 represented by formula (Ia): 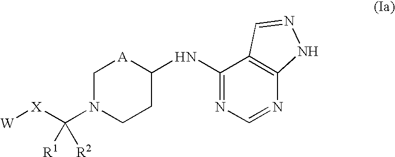
and/or an individual enantiomer, diastereomer, or a pharmaceutically acceptable salt thereof, wherein
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;
X is C₁₋₃ alkyl optionally substituted with one or more substituents selected from the group consisting of hydrogen, halogen, hydroxyl and (═O);
R¹ and R² each is independently selected from the group consisting of hydrogen and C₁₋₃ alkyl; and
A is a bond or C₁₋₃ alkyl, optionally substituted with one or more substituents selected from the group consisting of hydrogen and halogen.

4. A compound of claim 1 represented by Formula (Ib): 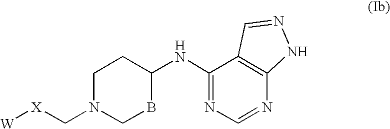
and/or an individual enantiomer, diastereomer, or a pharmaceutically acceptable salt thereof, wherein:
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;
X is C₁₋₃ alkyl optionally substituted with one or more substituents selected from the group consisting of hydrogen, halogen, hydroxyl and (═O); and
B is C₁alkyl, optionally substituted with one or more substituents selected from the group consisting of hydrogen and halogen.

5. A compound of claim 1 represented by Formula (Ic): 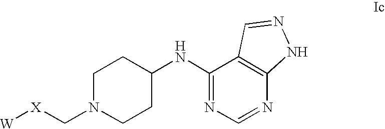
and/or an individual enantiomer, diastereomer, or a pharmaceutically acceptable salt thereof, wherein:
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl; and
X is C₁₋₃ alkyl optionally substituted with one or more substituents selected from the group consisting of hydrogen, halogen, hydroxyl and (═O).

6. A compound of claim 1 represented by Formula (Id): 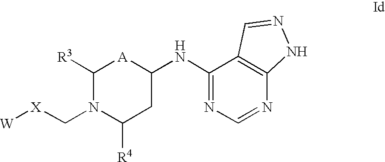
and/or an individual enantiomer, stereoisomer, or a pharmaceutically acceptable salt thereof, wherein:
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;
X is C₁₋₃ alkyl optionally substituted with one or more substituents selected from the group consisting of hydrogen and halogen;
A is a bond or C₁₋₃ alkyl, optionally substituted with one or more substituents selected from the group consisting of hydrogen and halogen; and
R³ and R⁴ each is independently selected from the group consisting of hydrogen, hydroxyl, cyano, and C₁₋₃ alkyl, wherein said alkyl is optionally substituted with one or more substituents selected from the group consisting of halogen, hydroxyl, C₁₋₄ alkoxy, and cyano,
where R³ and R⁴, along with the ring to which they are attached, optionally can join to form a bridged cycloalkyl.

7. A compound of claim 1 represented by Formula (Ie): 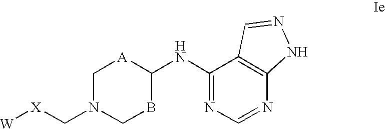
and/or an individual enantiomer, stereoisomer, or a pharmaceutically acceptable salt thereof, wherein:
W is aryl, optionally substituted with 1-5 substituents independently selected from the group consisting of halogen, C₃₋₆ cycloalkyl, C₁₋₄ alkoxy, and C₁₋₆ alkyl, wherein said alkyl is optionally substituted with one or more substituents selected from the group consisting of hydrogen, halogen and hydroxyl;
X is C₁₋₃ alkyl optionally substituted with one or more substituents selected from the group consisting of hydrogen, halogen, hydroxyl and (═O);
A is a bond or C₁₋₃ alkyl, optionally substituted with one or more substituents selected from the group consisting of hydrogen and halogen; and
B is C₁alkyl, optionally substituted with one or more substituents selected from the group consisting of hydrogen and halogen, where a ring is formed comprising A and B, where an individual carbon atom in A and an individual carbon atom in B optionally can join to bridge said ring.

8. A compound of claim 1 selected from: 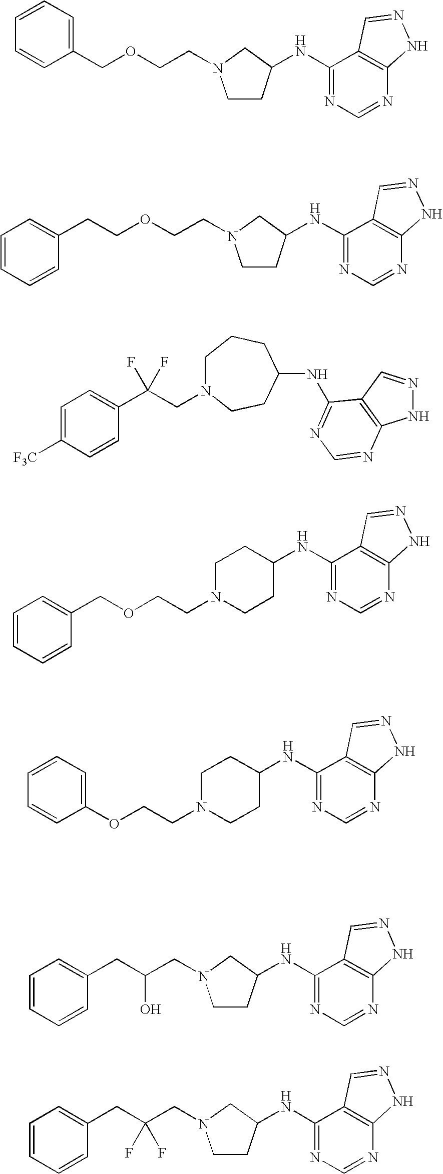 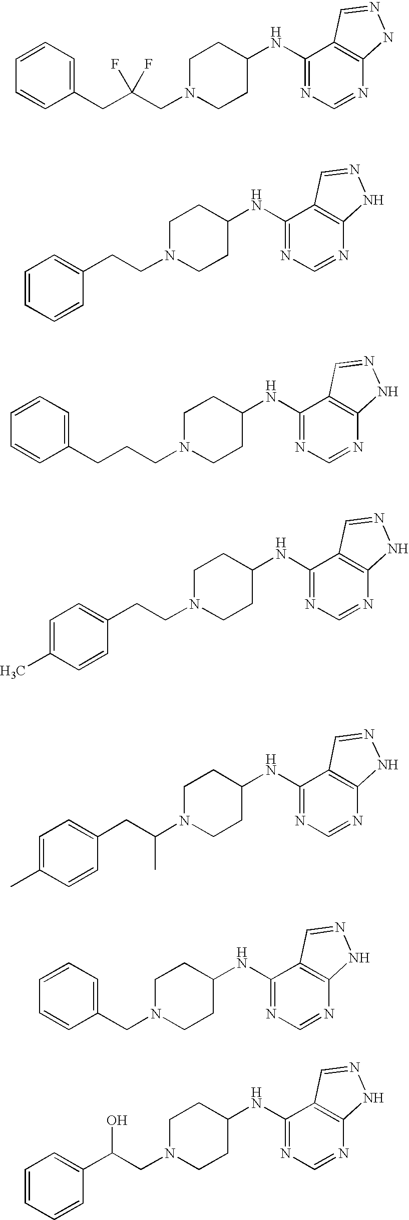 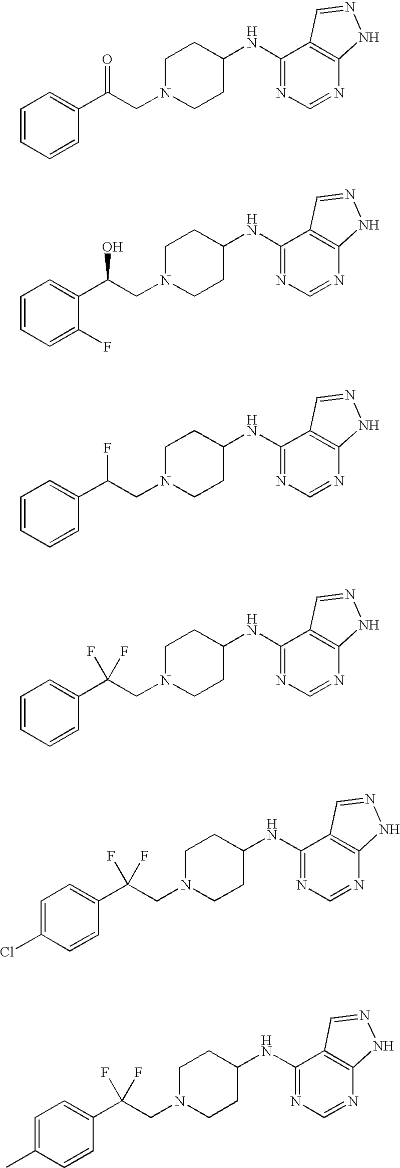 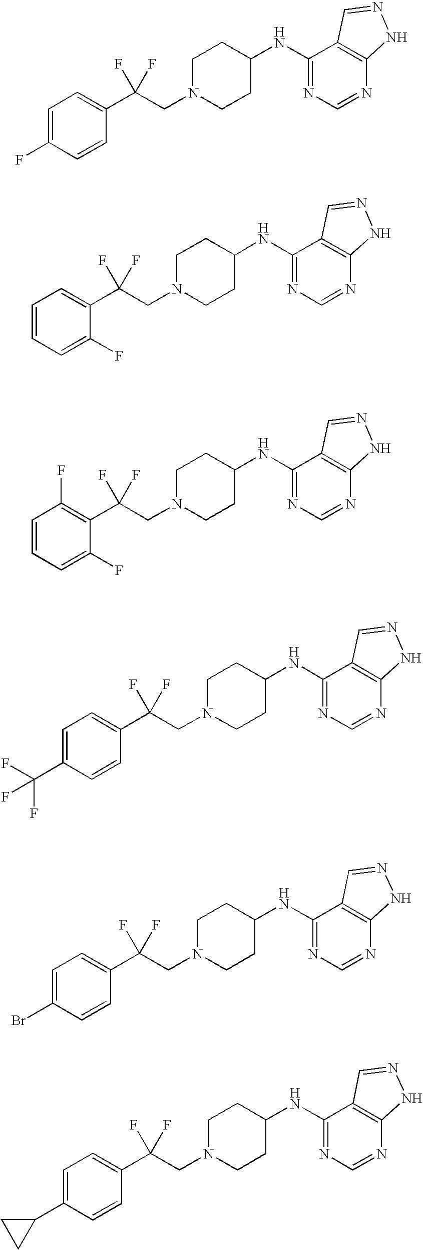 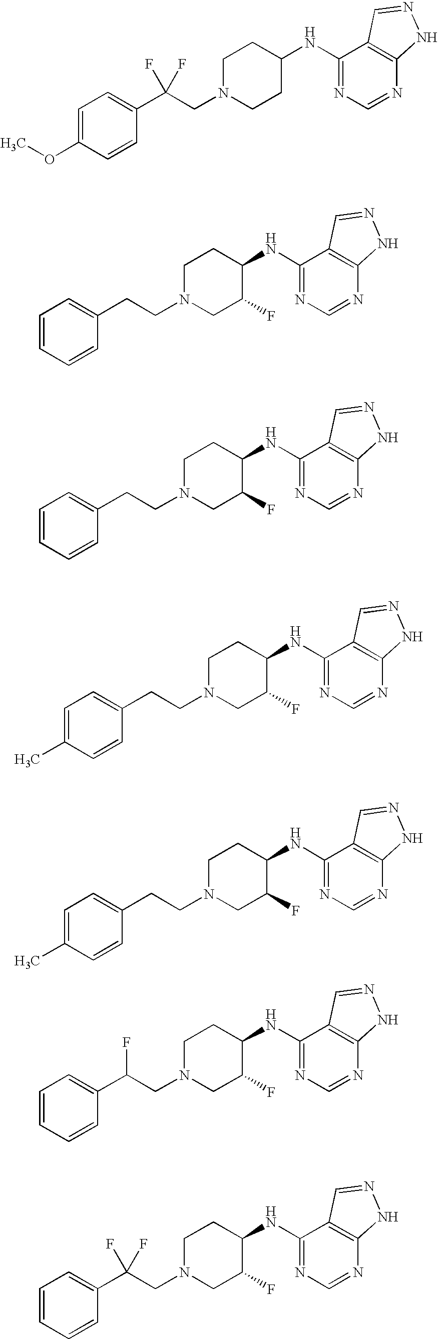 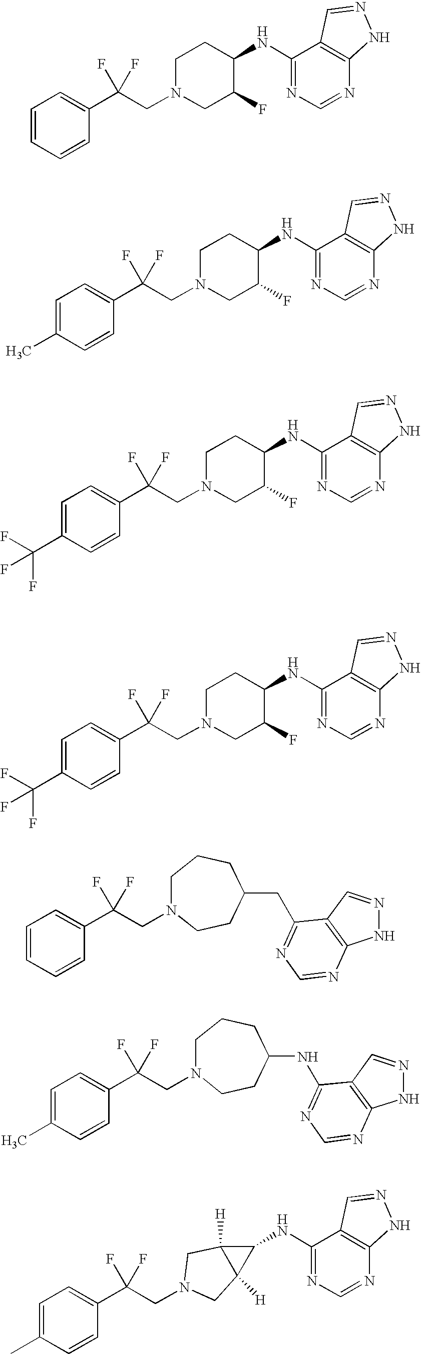 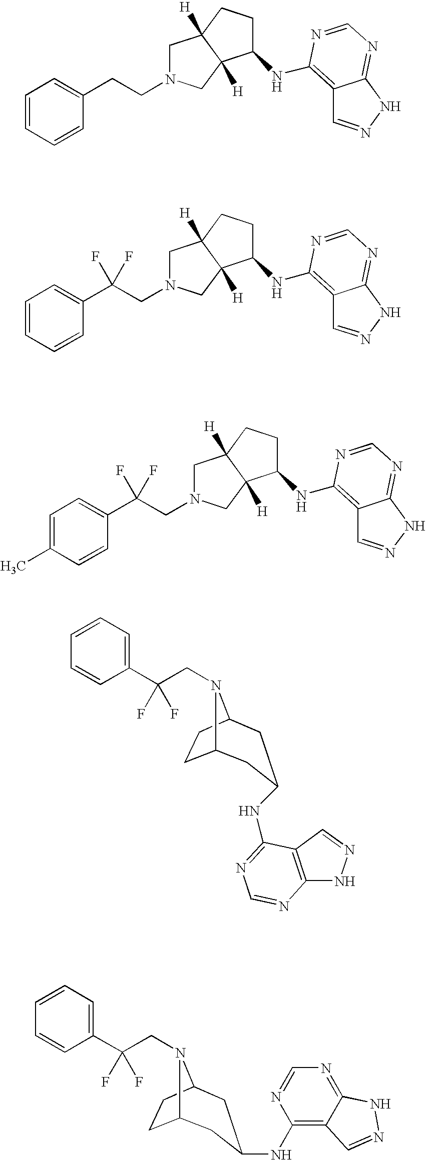 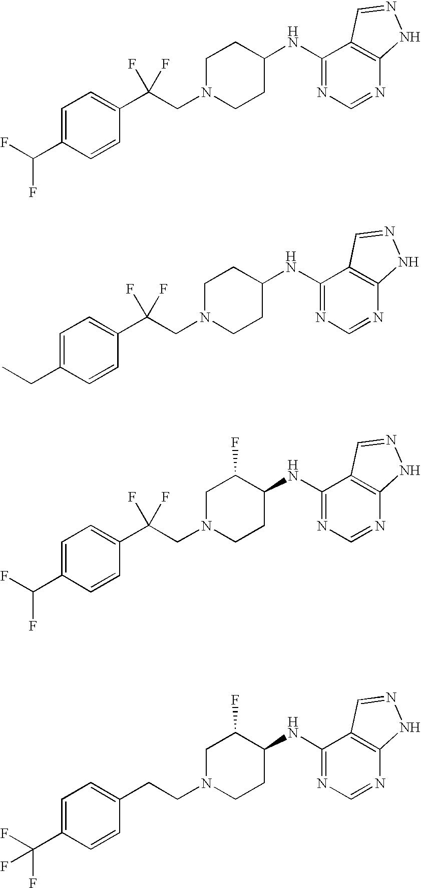
and/or an individual enantiomer, diastereomer, or a pharmaceutically acceptable salt thereof.

9. A pharmaceutical composition comprising an inert carrier and a therapeutically effective amount of a compound according to claim 1.

10. The pharmaceutical composition according to claim 9, further comprising a second therapeutic agent selected from the group consisting of: (i) non-steroidal anti-inflammatory agents; (ii) COX-2 inhibitors; (iii) bradykinin B1 receptor antagonists; (iv) sodium channel blockers and antagonists; (v) nitric oxide synthase (NOS) inhibitors; (vi) glycine site antagonists; (vii) potassium channel openers; (viii) AMPA/kainate receptor antagonists; (ix) calcium channel antagonists; (x) GABA-A receptor modulators (e.g., a GABA-A receptor agonist); (xi) matrix metalloprotease (MMP) inhibitors; (xii) thrombolytic agents; (xiii) opioids such as morphine; (xiv) neutrophil inhibitory factor (NIF); (xv) L-Dopa; (xvi) carbidopa; (xvii) levodopa/carbidopa; (xviii) dopamine agonists such as bromocriptine, pergolide, pramipexole, ropinirole; (xix) anticholinergics; (xx) amantadine; (xxi) carbidopa; (xxii) catechol O-methyltransferase (“COMT”) inhibitors such as entacapone and tolcapone; (xxiii) Monoamine oxidase B (“MAO-B”) inhibitors; (xiv) opiate agonists or antagonists; (xv) 5HT receptor agonists or antagonists; (xvi) NMDA receptor agonists or antagonists; (xvii) NK1 antagonists; (xviii) selective serotonin reuptake inhibitors (“SSRI”) and/or selective serotonin and norepinephrine reuptake inhibitors (“SSNRI”); (xxix) tricyclic antidepressant drugs, (xxx) norepinephrine modulators; (xxxi) lithium; (xxxii) valproate; and (xxxiii) neurontin (gabapentin).

11-12. (canceled)

13. A method for treating or preventing pain, Parkinson's disease, Alzheimer's disease, epilepsy, depression, anxiety, ischemic brain injury including stroke in a patient in need thereof comprising administering to said patient a therapeutically effective amount, or a prophylactically effective amount, of a compound according to claim 1, or a pharmaceutically acceptable salt thereof.

14. A method of claim 13 for treating or preventing chronic, visceral, inflammatory and neuropathic pain syndromes in a patient in need thereof comprising administering to said patient a therapeutically effective amount, or a prophylactically effective amount, of a compound according to claim 1, or a pharmaceutically acceptable salt thereof.

15. A method of claim 13 for treating or preventing pain resulting from, or associated with, traumatic nerve injury, nerve compression or entrapment, postherpetic neuralgia, trigeminal neuralgia, diabetic neuropathy, cancer and chemotherapy, in a patient in need thereof comprising administering to said patient a therapeutically effective amount, or a prophylactically effective amount, of a compound according to claim 1, or a pharmaceutically acceptable salt thereof.

16. A method of claim 13 for treating or preventing chronic lower back pain in a patient in need thereof comprising administering to said patient a therapeutically effective amount, or a prophylactically effective amount, of a compound according to claim 1, or a pharmaceutically acceptable salt thereof.

17. A method of claim 13 for treating or preventing phantom limb pain in a patient in need thereof comprising administering to said patient a therapeutically effective amount, or a prophylactically effective amount, of a compound according to claim 1, or a pharmaceutically acceptable salt thereof.

18. A method of claim 13 for treating or preventing HIV- and HIV treatment-induced neuropathy, chronic pelvic pain, neuroma pain, complex regional pain syndrome, chronic arthritic pain and related neuralgias in a patient in need thereof comprising administering to said patient a therapeutically effective amount, or a prophylactically effective amount, of a compound according to claim 1, or a pharmaceutically acceptable salt thereof.

19. A method of claim 13 for treating or preventing epilepsy and partial and generalized tonic seizures in a patient in need thereof comprising administering to said patient a therapeutically effective amount, or a prophylactically effective amount, of a compound according to claim 1, or a pharmaceutically acceptable salt thereof.
